(19) United States
(12) Patent Application Publication
(10) Pub. No.: US 2024/0239876 A1
(43) Pub. Date: Jul. 18, 2024

(54) ANTI-HUMAN IMMUNODEFICIENCY VIRUS-1 ANTIBODIES AND METHODS FOR USES THEREOF

(71) Applicant: GRIFOLS DIAGNOSTIC SOLUTIONS INC, Emeryville, CA (US)

(72) Inventors: ELISABETE NASCIMENTO, Emeryville, CA (US); JODY MELTON WITT, Emeryville, CA (US)

(21) Appl. No.: 18/015,317

(22) PCT Filed: Jul. 13, 2021
(86) PCT No.: PCT/IB2021/056284

§ 371 Date: Jan. 9, 2023

Related U.S. Application Data
(60) Provisional application No. 63/051,323, filed on Jul. 13, 2020.

(51) Int. Cl. C07K 16/10; G01N 33/543; G01N 33/569

(52) U.S. Cl. CPC C07K 16/1054; G01N 33/543; G01N 33/56988; C07K 2317/52; C07K 2317/565; G01N 2333/161

(57) ABSTRACT

An anti-HIV-1 antibody comprising L-CDR1, L-CDR2 and L-CDR3, wherein L-CDR1 is selected from the group consisting of SEQ ID NO: 15, SEQ ID NO: 18, SEQ ID NO: 21, and a sequence that differs from anyone of SEQ ID NOs: 15, 18, or 21 by one or two substitutions, deletions, or additions, the amino acid sequence of L-CDR2 is selected from the group consisting of SEQ ID NO: 16, SEQ ID NO: 19, SEQ ID NO: 22, and a sequence that differs from anyone of SEQ ID NOs: 16, 19, or 22 by one or two substitutions, deletions, or additions, and the amino acid sequence of L-CDR3 is selected from the group consisting of SEQ ID NO: 17, SEQ ID NO: 20, SEQ ID NO: 23, and a sequence that differs from anyone of SEQ ID NOs: 17, 20, or 23 by one or two substitutions, deletions, or additions.

### REFERENCE TO SEQUENCE LISTING

The present application is being filed along with a Sequence Listing in electronic format. A Replacement Electronic Sequence Listing is provided as a file entitled HFG039001APCREPLACEMENTSEQUENCELISTING.txt, which is 48,553 bytes in size and was created on Sep. 11, 2023. The information in the electronic format of the replacement Sequence Listing is incorporated herein by reference in its entirety.

### TECHNICAL FIELD

This application relates to antibodies against Human Immunodeficiency Virus-1 (anti-HIV-1) that specifically bind to HIV-1 p24 protein. The present invention also refers to methods and assays for detection of HIV-1 in samples using said antibodies.

### BACKGROUND

The human immunodeficiency virus 1 (HIV-1) is a retrovirus that infects 37.9 million people worldwide, killing around 1 million individuals every year, particularly in vulnerable populations unable to access diagnosis and treatment (Soliman, M., et al. Mechanisms of HIV Control. Current HIV/AIDS Reports 2017, vol. 14(3); 101-9). HIV-1 is the leading cause of acquired immune deficiency syndrome (AIDS), an incurable disease transmitted through sexual contact from HIV-1 infected individuals or by exposure to blood or blood-derived contaminated products. The virus targets the immune system by destroying and impairing the function of immune cells. Infected individuals become immunodeficient and susceptible to other opportunistic infections as well as some types of cancer (WHO website source—https://www.who.int/news-room/fact-sheets/detail/hiv-aids). Currently, only 46% of HIV-1 infected individuals know their infectious status. Hence, detection of HIV-1 in acute infection is a critical public health concern (Stone, M., et al. Comparison of detection limits of fourth- and fifth-generation combination HIV antigen-antibody, p24 antigen, and viral load assays on diverse HIV isolates. Journal of Clinical Microbiology 2018, vol. 56(8); 1-12).

In this particular context, the goal is to diagnose HIV-1 in the weeks immediately after an individual contracted the infection (acute phase) as this will likely prevent secondary transmission and allow for early access to treatment and care (Lewis J., et al. Field accuracy of fourth-generation rapid diagnostic tests for acute HIV-1: a systematic review. AIDS 2015, vol. 29(18); 2465-71). To achieve this goal in a timely manner, the use of early biomarkers for HIV-1 detection is key. Among the most commonly used biomarkers for diagnosis of HIV-1 infection are antibodies against the viral structural proteins. Here, p24 is considered an important biomarker for early HIV-1 detection, as it is the most abundant structural protein of the HIV-1 viral envelope and it is secreted at high levels in the blood serum during the initial stages of infection. p24 is a polymerized capsid protein that acts as the major structural component of the HIV-1 envelope around the viral RNA molecule. p24 is a 24-25 kDa protein derived from a Gag polyprotein precursor that, like the HIV-1 RNA, can be detected before seroconversion (Gray, E. R., et al. p24 revisited: a landscape review of antigen detection for early HIV diagnosis. AIDS. 2018, vol. 32(15); 2089-102).

Present guidelines from the Centers for Disease Control and Prevention (CDC) and the World Health Organization (WHO) recommend the use of fourth generation antibody-antigen assays as a preferred method for HIV-1 screening. These tests detect p24 antigen and anti-HIV-1 antibodies and have narrowed the diagnostic window period from 4 to 2 weeks post-exposure (Gray, E. R., et al. p24 revisited: a landscape review of antigen detection for early HIV diagnosis. AIDS. 2018, vol. 32(15); 2089-102; Codoner, F., et al. Gag protease coevolution analyses define novel structural surfaces in the HIV-1 matrix and capsid involved in resistance to Protease Inhibitors. Scientific Reports 2017, vol. 7(3717); 1-10; Alexander T S. Human Immunodeficiency Virus Diagnostic Testing: 30 Years of Evolution. Clinical and Vaccine Immunology 2016, vol. 23(4); 249-53; WHO. World Health Organization Model List of Essential In Vitro Diagnostics. 1st ed. Geneva. 2018; Centers for Disease Control and Prevention. 2017. National HIV testing day and new testing recommendations. Morbidity and Mortality Weekly Report vol. 63(25); 537-37).

However, there is still a need for anti HIV-1 antibodies that specifically bind to p24 antigen with high binding capacity and good manufacturing characteristics, since some antibodies currently commercially available show low sensitivity for early p24 detection.

Thus, the present invention provides anti-HIV-1 antibodies having improved binding capacity to HIV-1 p24 protein when compared to similar commercial reagents. These antibodies recognize novel, non-cross-reactive epitopes and can be used as single entities or as capture/detection partners in multiple HIV-1 immunoassays, such as immunodiagnostic or blood screening platforms.

### SUMMARY

Where the present specification speaks to the sequence of CDR X or a sequence that differs from CDR X by one or two substitutions, deletions, or additions it is to be understood that such substitutions, deletions, or additions can arise at any amino acid of the range of amino acids defined by CDR X. The present specification individualizes each particular amino acid within the range of amino acids defined by CDR X as being suitable for such substitutions, deletions, or additions.

As a non-limiting illustration, L-CDR1 for Antibody #A can be defined as comprising the sequence of amino acids (1)-(11), RASQDISNYLH [as outlined in SEQ ID NO: 15]. Each of positions 1, 2, 3, 4, 5, 6, 7, 8, 9, 10, and 11 is to be considered suitable for a substitution, deletion, or addition unless otherwise specified or later refined by amendment.

In a first aspect, the present invention discloses an anti-HIV-1 antibody comprising a light chain comprising complementary determining regions L-CDR1, L-CDR2 and L-CDR3, wherein the amino acid sequence of L-CDR1 is selected from the group consisting of SEQ ID NO: 15, SEQ ID NO: 18, SEQ ID NO: 21, and a sequence that differs from anyone of SEQ ID NO: 15, 18, or 21 by one or two substitutions, deletions, or additions, the amino acid sequence of L-CDR2 is selected from the group consisting of SEQ ID NO: 16, SEQ ID NO: 19, SEQ ID NO: 22, and a sequence that differs from anyone of SEQ ID NO: 16, 19, or 22 by one or two substitutions, deletions, or additions, and the amino acid sequence of L-CDR3 is selected from the group consisting of SEQ ID NO: 17, SEQ ID NO: 20, SEQ ID NO: 23, and a sequence that differs from anyone of SEQ ID NO: 17, 20, or 23 by one or two substitutions, deletions, or additions.

In other embodiments, the anti-HIV-1 antibody of the present invention comprises a heavy chain comprising complementary determining regions H-CDR1, H-CDR2 and H-CDR3, wherein the amino acid sequence of H-CDR1 is selected from the group consisting of SEQ ID NO: 24, SEQ ID NO: 27, SEQ ID NO: 30, and a sequence that differs from anyone of SEQ ID NO: 24, 27, or 30 by one or two substitutions, deletions, or additions, the amino acid sequence of H-CDR2 is selected from the group consisting of SEQ ID NO: 25, SEQ ID NO: 28, SEQ ID NO: 31, and a sequence that differs from anyone of SEQ ID NO: 25, 28, or 31 by one or two substitutions, deletions, or additions, and the amino acid sequence of H-CDR3 is selected from the group consisting of SEQ ID NO: 26, SEQ ID NO: 29, SEQ ID NO: 32, and a sequence that differs from anyone of SEQ ID NO: 26, 29, or 32 by one or two substitutions, deletions, or additions.

In some embodiments, the light chain of the anti-HIV-1 antibody of the present invention comprises a sequence having about 90% homology with the amino acid sequence of SEQ ID NO: 7, or SEQ ID NO: 8, or SEQ ID NO: 9. In other embodiments, the light chain comprises the amino acid sequence of SEQ ID NO: 7, or SEQ ID NO: 8, or SEQ ID NO: 9.

In some embodiments, the heavy chain of the anti-HIV-1 antibody of the present invention comprises a sequence having about 90% homology with the amino acid sequence of SEQ ID NO: 10, SEQ ID NO: 11, or SEQ ID NO: 12. In other embodiments, the heavy chain comprises the amino acid sequence of SEQ ID NO: 10, SEQ ID NO: 11, or SEQ ID NO: 12.

In some embodiments, the amino acid sequence of L-CDR1 comprises SEQ ID NO: 15, or a sequence that differs from SEQ ID NO: 15 by one or two substitutions, deletions, or additions, the amino acid sequence of L-CDR2 comprises SEQ ID NO: 16, or a sequence that differs from SEQ ID NO: 16 by one or two substitutions, deletions, or additions, and the amino acid sequence of L-CDR3 comprises SEQ ID NO: 17, or a sequence that differs from SEQ ID NO: 17 by one or two substitutions, deletions, or additions. In other embodiments, the amino acid sequence of L-CDR1 comprises SEQ ID NO: 18, or a sequence that differs from SEQ ID NO: 18 by one or two substitutions, deletions, or additions, the amino acid sequence of L-CDR2 comprises SEQ ID NO: 19, or a sequence that differs from SEQ ID NO: 19 by one or two substitutions, deletions, or additions, and the amino acid sequence of L-CDR3 is SEQ ID NO: 20 or a sequence that differs from SEQ ID NO: 20 by one or two substitutions, deletions, or additions. In other embodiments, the amino acid sequence of L-CDR1 comprises SEQ ID NO: 21, or a sequence that differs from SEQ ID NO: 21 by one or two substitutions, deletions, or additions, the amino acid sequence of L-CDR2 comprises SEQ ID NO: 22, or a sequence that differs from SEQ ID NO: 22 by one or two substitutions, deletions, or additions, and the amino acid sequence of L-CDR3 comprises SEQ ID NO: 23 or a sequence that differs from SEQ ID NO: 23 by one or two substitutions, deletions, or additions.

In some embodiments, the amino acid sequence of H-CDR1 comprises SEQ ID NO: 24, or a sequence that differs from SEQ ID NO: 24 by one or two substitutions, deletions, or additions, the amino acid sequence of H-CDR2 comprises SEQ ID NO: 25, or a sequence that differs from SEQ ID NO: 25 by one or two substitutions, deletions, or additions, and the amino acid sequence of H-CDR3 comprises SEQ ID NO: 26, or a sequence that differs from SEQ ID NO: 26 by one or two substitutions, deletions, or additions. In other embodiments, the amino acid sequence of H-CDR1 comprises SEQ ID NO: 27, or a sequence that differs from SEQ ID NO: 27 by one or two substitutions, deletions, or additions, the amino acid sequence of H-CDR2 comprises SEQ ID NO: 28, or a sequence that differs from SEQ ID NO: 28 by one or two substitutions, deletions, or additions, and the amino acid sequence of H-CDR3 comprises SEQ ID NO: 29 or a sequence that differs from SEQ ID NO: 29 by one or two substitutions, deletions, or additions. In other embodiments, the amino acid sequence of H-CDR1 comprises SEQ ID NO: 30, or a sequence that differs from SEQ ID NO: 30 by one or two substitutions, deletions, or additions, the amino acid sequence of H-CDR2 comprises SEQ ID NO: 31, or a sequence that differs from SEQ ID NO: 31 by one or two substitutions, deletions, or additions, and the amino acid sequence of H-CDR3 comprises SEQ ID NO: 32 or a sequence that differs from SEQ ID NO: 32 by one or two substitutions, deletions, or additions.

In some embodiments, the amino acid sequence of L-CDR1 comprises SEQ ID NO: 15 or a sequence that differs from SEQ ID NO: 15 by one or two substitutions, deletions, or additions, the amino acid sequence of L-CDR2 comprises SEQ ID NO: 16 or a sequence that differs from SEQ ID NO: 16 by one or two substitutions, deletions, or additions, the amino acid sequence of L-CDR3 comprises SEQ ID NO: 17 or a sequence that differs from SEQ ID NO: 17 by one or two substitutions, deletions, or additions, the amino acid sequence of H-CDR1 comprises SEQ ID NO: 24 or a sequence that differs from SEQ ID NO: 24 by one or two substitutions, deletions, or additions, the amino acid sequence of H-CDR2 comprises SEQ ID NO: 25 or a sequence that differs from SEQ ID NO: 25 by one or two substitutions, deletions, or additions, and the amino acid sequence of H-CDR3 comprises SEQ ID NO: 26 or a sequence that differs from SEQ ID NO: 26 by one or two substitutions, deletions, or additions.

In some embodiments, the amino acid sequence of L-CDR1 comprises SEQ ID NO: 18 or a sequence that differs from SEQ ID NO: 18 by one or two substitutions, deletions, or additions, the amino acid sequence of L-CDR2 comprises SEQ ID NO: 19 or a sequence that differs from SEQ ID NO: 19 by one or two substitutions, deletions, or additions, the amino acid sequence of L-CDR3 comprises SEQ ID NO: 20 or a sequence that differs from SEQ ID NO: 20 by one or two substitutions, deletions, or additions, the amino acid sequence of H-CDR1 comprises SEQ ID NO: 27 or a sequence that differs from SEQ ID NO: 27 by one or two substitutions, deletions, or additions, the amino acid sequence of H-CDR2 comprises SEQ ID NO: 28 or a sequence that differs from SEQ ID NO: 28 by one or two substitutions, deletions, or additions, and the amino acid sequence of H-CDR3 comprises SEQ ID NO: 29 or a sequence that differs from SEQ ID NO: 29 by one or two substitutions, deletions, or additions.

In some embodiments, the amino acid sequence of L-CDR1 comprises SEQ ID NO: 21 or a sequence that differs from SEQ ID NO: 21 by one or two substitutions, deletions, or additions, the amino acid sequence of L-CDR2 comprises SEQ ID NO: 22 or a sequence that differs from SEQ ID NO: 22 by one or two substitutions, deletions, or additions, the amino acid sequence of L-CDR3 comprises SEQ ID NO: 23. or a sequence that differs from SEQ ID NO: 23 by one or two substitutions, deletions, or additions, the amino acid sequence of H-CDR1 comprises SEQ ID NO: 30 or a sequence that differs from SEQ ID NO: 30 by one or two substitutions, deletions, or additions, the amino acid sequence of H-CDR2 comprises SEQ ID NO: 31 or a sequence that differs from SEQ ID NO: 31 by one or two substitutions, deletions, or additions, and the amino acid sequence of H-CDR3 comprises SEQ ID NO: 32 or a sequence that differs from SEQ ID NO: 32 by one or two substitutions, deletions, or additions.

In some embodiments, the light chain of the anti-HIV-1 antibody of the present invention comprises the amino acid sequence selected from the group consisting of SEQ ID NO: 1, SEQ ID NO: 2, or SEQ ID NO: 3.

In some embodiments, the heavy chain of the anti-HIV-1 antibody of the present invention comprises the amino acid sequence selected from the group consisting of SEQ ID NO: 4, SEQ ID NO: 5, or SEQ ID NO: 6.

In some embodiments, the anti-HIV-1 antibody of the present invention specifically binds to an epitope of HIV-1 p24 protein comprising the amino acid sequence of SEQ ID NO: 33.

In some embodiments, the amino acid sequence of L-CDR1 of the anti-HIV-1 antibody of the present invention comprises SEQ ID NO: 21 or a sequence that differs from SEQ ID NO: 21 by one or two substitutions, deletions, or additions, the amino acid sequence of L-CDR2 comprises SEQ ID NO: 22 or a sequence that differs from SEQ ID NO: 22 by one or two substitutions, deletions, or additions, and the amino acid sequence of L-CDR3 comprises SEQ ID NO: 23. or a sequence that differs from SEQ ID NO: 23 by one or two substitutions, deletions, or additions.

In some embodiments, the amino acid sequence of H-CDR1 of the anti-HIV-1 antibody of the present invention comprises SEQ ID NO: 30 or a sequence that differs from SEQ ID NO: 30 by one or two substitutions, deletions, or additions, the amino acid sequence of H-CDR2 comprises SEQ ID NO: 31 or a sequence that differs from SEQ ID NO: 31 by one or two substitutions, deletions, or additions, and the amino acid sequence of H-CDR3 comprises SEQ ID NO: 32 or a sequence that differs from SEQ ID NO: 32 by one or two substitutions, deletions, or additions.

In some preferred embodiments, the light chain of said antibody comprises the amino acid sequence selected from the group consisting of SEQ ID NO: 1, SEQ ID NO: 2, or SEQ ID NO: 3 and the heavy chain of said antibody comprises the amino acid sequence selected from the group consisting of SEQ ID NO: 4, SEQ ID NO: 5, or SEQ ID NO: 6.

In some embodiments, the anti-HIV-1 antibody of the present invention is a monoclonal antibody or a recombinant antibody. In other embodiments, said antibody is an antibody fragment. When the anti-HIV-1 antibody is an antibody fragment, it is selected from variable fragments (Fv), single-chain Fvs (scFv), bispecific antibodies (sc(Fv)2), single chain antibodies, single domain antibodies, Fab fragments, F(ab′)2 fragments, Fab′ fragments, disulfide-linked Fv (dsFv), chemically conjugated Fv (ccFv), diabodies, anti-idiotypic (anti-Id) antibodies, affibodies, nanobodies, and unibodies.

In some embodiments, the anti-HIV-1 antibody comprises a constant region of the murine IgG1 class or the murine IgG2a class.

In some embodiments the anti-HIV-1 antibody is bound to a solid support.

In some aspects, the present invention discloses a cell comprising the anti-HIV-1 antibody of the present invention.

In other aspects, the present invention discloses a nucleic acid comprising a nucleotide sequence encoding the anti-HIV-1 antibody, a promoter operably linked to the nucleotide sequence and a selectable marker. A cell comprising said nucleic acid is also disclosed herein.

The present invention also discloses compositions comprising the anti-HIV-1 antibody as described herein, and a solid support, wherein the anti-HIV-1 antibody is covalently or non-covalently bound to the solid support. In some embodiments, the solid support comprises a particle, a bead, a membrane, a surface, a polypeptide chip, a microtiter plate, or the solid-phase of a chromatography column.

The present invention also discloses kits for detecting the presence of HIV-1 in a sample, said kit comprising at least one anti-HIV-1 antibody according to the present invention and a solid support, wherein said at least one antibody is covalently or non-covalently bound to a solid support.

### BRIEF DESCRIPTION OF THE DRAWINGS

FIG. 1. SE-UPLC analysis % monomer for antibody #A and SDS-PAGE for antibody #A single clones (lanes 1, 2 and 3 represent subclones ran in reducing and non-reducing conditions, respectively).

FIG. 2. SE-UPLC analysis % monomer for antibody #B and SDS-PAGE for antibody #B single clones (lanes 1, 2 and 3 represent subclones ran in reducing and non-reducing conditions, respectively).

FIG. 3. SE-UPLC analysis % monomer for antibody #D and SDS-PAGE for antibody #D single clones (lanes 1, 2 and 3 represent subclones ran in reducing and non-reducing conditions, respectively).

FIG. 4. PDB predicted structure for antibodies #A, #B and #D (4A, 4B and 4D respectively). For antibody #A the PDB structure code 2XKN was used in the homology query, while codes 5OPY and 1F3D were used respectively for antibodies B # and #D, respectively.

FIG. 5. Sensorgram of saturating antibody #A versus competing #B and #D. Antibodies #B and #D add signal to #A demonstrating these antibodies do not compete for binding within the same epitope region.

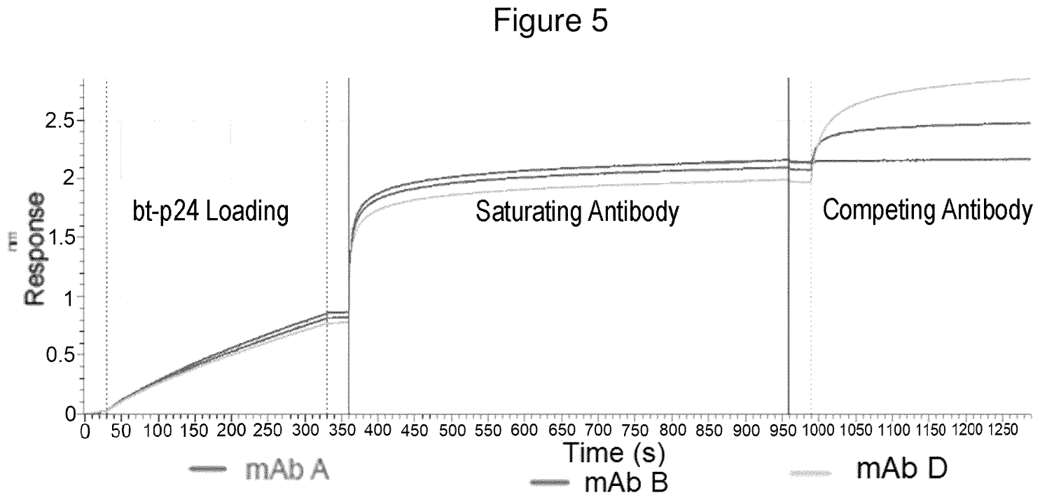

FIG. 6. Sensorgram of saturating antibody #B versus competing #A and #D. Antibodies #A and #D add signal to #B demonstrating that these antibodies do not compete for binding within the same epitope region.

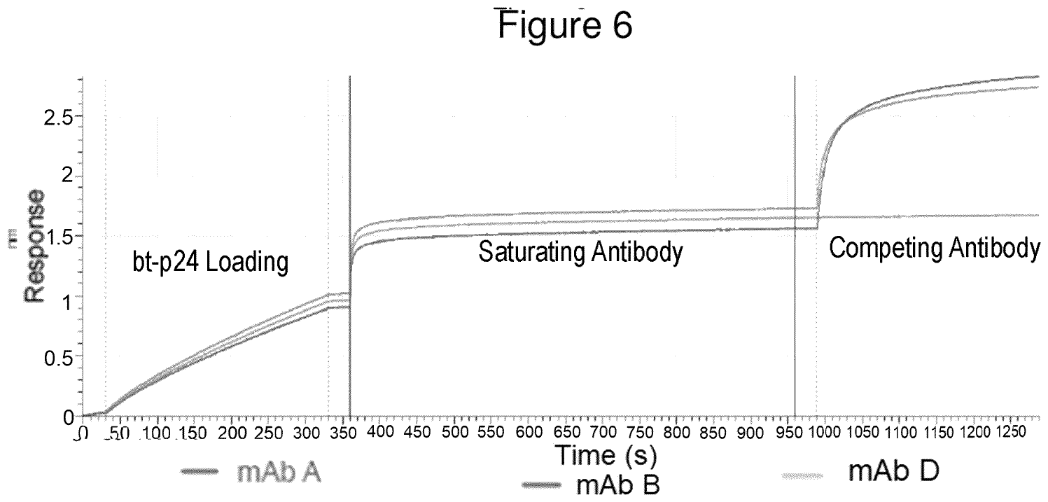

FIG. 7. Sensorgram of saturating antibody #D versus competing #A and #B. Antibodies #A and #B add signal to #D demonstrating that these antibodies do not compete for binding within the same epitope region.

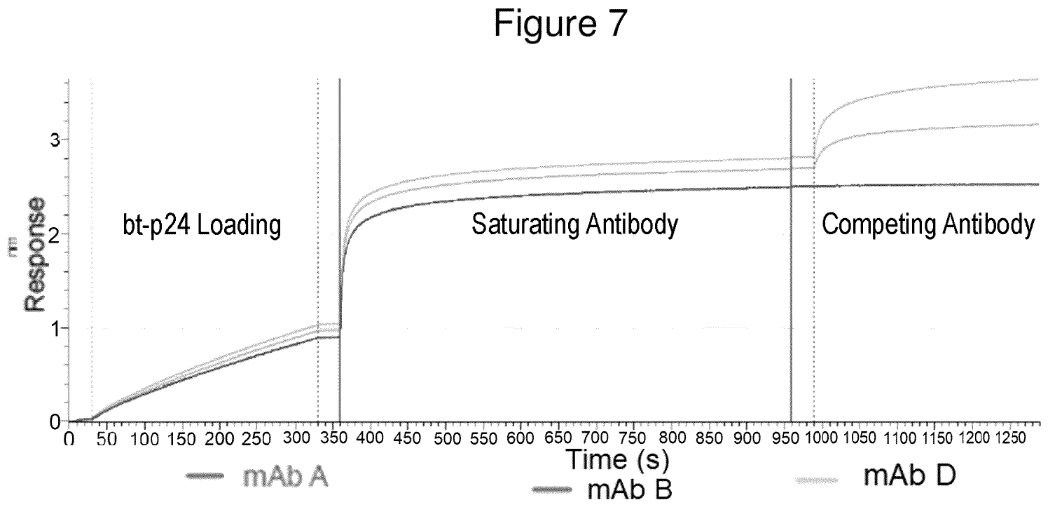

FIG. 8 Sensorgram of antibodies #A, #B and #D association to HIV-1 p24 in the absence of competing antibody. Each antibody attains its full binding signal (experimental control).

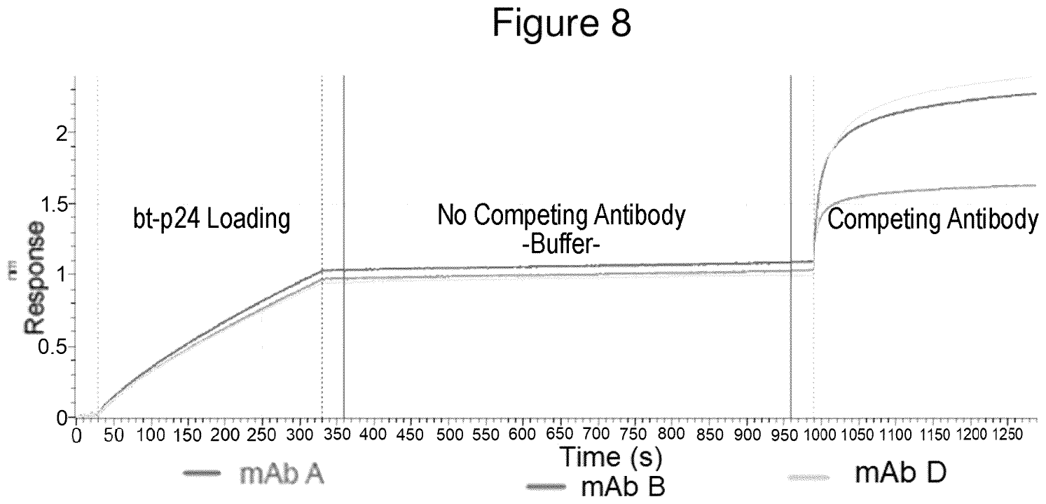

FIG. 9. Binding kinetics of antibodies #A, #B and #D and commercial mAb #1 to antigen HIV-1 p24 calculated by Biolayer Interferometry (BLI). Sensorgrams were performed for gradient concentrations of 0.1-33 nM and were fitted with a 1:1 binding model in order to calculate ka (association rate constant), kd (dissociation rate constant) and KD (equilibrium dissociation constant).

FIG. 10. Binding of antibodies #A, #B and #D and commercial mAb #2 to HIV-1 p24 capsid protein by indirect ELISA. Titration curves for each antibody start at a concentration of 2 μg/mL, with subsequent 1:10 dilutions (Left). Signal-to-noise data for an antibody concentration of 200 ng/ml is shown on the right, demonstrating poor performance of commercial mAb #2 when compared to antibodies #A, #B and #D.

### DETAILED DESCRIPTION

The following description is merely intended to illustrate various embodiments of the present disclosure. As such, the specific modifications discussed are not intended to be limiting. It will be apparent to one skilled in the art that various equivalents, changes, and modifications may be made without departing from the spirit or scope of the subject matters presented herein, and it is understood that such equivalent embodiments are to be included herein.

As used in this specification and the appended claims, the singular forms “a,” “an” and “the” include plural references unless the content clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used and will be apparent to those of skill in the art. All publications and other references mentioned herein are incorporated by reference in their entirety. In case of conflict, the present specification, including definitions, will control. The materials, methods, and examples are illustrative only and not intended to be limiting.

Each embodiment in this specification is to be applied mutatis mutandis to every other embodiment unless expressly stated otherwise.

The following terms, unless otherwise indicated, shall be understood to have the following meanings:

As used herein, the term “nucleic acid” refers to any materials comprised of DNA or RNA. Nucleic acids can be made synthetically or by living cells.

A “nucleotide,” as used herein, is a subunit of a nucleic acid consisting of a phosphate group, a 5-carbon sugar and a nitrogenous base. The 5-carbon sugar found in RNA is ribose. In DNA, the 5-carbon sugar is 2′-deoxyribose. The term also includes analogs of such subunits.

As used herein, the term “polynucleotide” refers to a polymeric chain of nucleotides. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native inter-nucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation.

As used herein, the term “protein” or refers to large biological molecules, or macromolecules, consisting of one or more chains of amino acid residues. Many proteins are enzymes that catalyze biochemical reactions and are vital to metabolism. Proteins also have structural or mechanical functions, such as actin and myosin in muscle and the proteins in the cytoskeleton, which form a system of scaffolding that maintains cell shape. Other proteins are important in cell signalling, immune responses, cell adhesion, and the cell cycle. However, proteins may be completely artificial or recombinant, i.e., not existing naturally in a biological system.

As used herein, the term “polypeptide” refers to both naturally-occurring and non-naturally-occurring proteins, and fragments, mutants, derivatives and analogs thereof. A polypeptide may be monomeric or polymeric. A polypeptide may comprise a number of different domains (peptides) each of which has one or more distinct activities.

As used herein, the term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, the term “fusion protein” refers to proteins comprising two or more amino acid sequences that do not co-exist in naturally-occurring proteins. A fusion protein may comprise two or more amino acid sequences from the same or from different organisms. The two or more amino acid sequences of a fusion protein are typically in frame without stop codons between them and are typically translated from mRNA as part of the fusion protein.

The term “fusion protein” and the term “recombinant” when referring to a protein according to (3), can be used interchangeably herein.

The terms “antibody” or “immunoglobulin”, as used herein, have the same meaning, and are used equally in the present invention. The term “antibody” as used herein refers to immunoglobulin molecules and immunologically active portions of immunoglobulin molecules, i.e., molecules that contain an antigen binding site that specifically binds an antigen. As such, the term antibody encompasses not only whole antibody molecules, but also antibody fragments or derivatives.

In natural antibodies, two heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, normally includes six CDRs, comprising the CDR set from each of a heavy and a light chain V region. Framework Regions (FRs) refer to amino acid sequences interposed between CDRs.

CDR can be identified in accordance with the definitions of the Kabat, Chothia, the accumulation of both Kabat and Chothia, AbM, contact, IMGT unique numbering, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others (See, e.g., Chothia et al., Nature 342:877-883, 1989). Other approaches to CDR identification include the “AbM definition,” which is a compromise between Kabat and Chothia and is derived using Oxford Molecular's AbM antibody modeling software (now Accelrys0), the “contact definition” of CDRs based on observed antigen contacts, set forth in MacCallum et al., J. Mol. Biol., 262:732-745, 1996, or “IMGT unique numbering”, which relies on the high conservation of the structure of the variable region (see Lefranc, M.-P. Nucl. Acids Res., 33, D593-D597, 2005). In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, IMGT unique numbering and/or conformational definitions, unless otherwise specified.

Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. Unless otherwise indicated, all CDRs set forth herein are derived according to the Abysis database website as per the scheme indicated.

As used herein, the terms “antibody” include isolated antibodies, polyclonal antibodies, monoclonal antibodies, multispecific antibodies, human antibodies, humanized antibodies (fully or partially humanized), animal antibodies, recombinant antibodies, chimeric antibodies, and antibody fragments.

As used herein, the term “monoclonal antibody” or “mAb” refers to an antibody composition having a homogeneous antibody population that bind to the same epitope. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. The term also encompasses antibodies obtained by other methods for production of monoclonal antibodies known in the art, such as the establishment of eukaryotic cells lines by transient or stable transfection.

As used herein, the term “recombinant antibody” refers to an antibody that is expressed from a cell or cell line transfected with one or more expression vectors comprising the coding sequence of the antibody, where said coding sequence is not naturally associated with the cell. Recombinant antibodies or fragments thereof are prepared, expressed, created or isolated by any recombinant mean, as well known by the skilled person.

In some embodiments, a “recombinant antibody” may also be a “monoclonal antibody” when it derives from a homogeneous antibody population that binds to the same epitope.

Thus, the term “antibody fragments” as used herein, include but are not limited to variable fragments (Fv), single-chain Fvs (scFv), bispecific antibodies (sc(Fv)₂), single chain antibodies, single domain antibodies, Fab fragments, F(ab′)₂ fragments, Fab′ fragments, disulfide-linked Fv (dsFv), chemically conjugated Fv (ccFv), diabodies and anti-idiotypic (anti-Id) antibodies, and functionally active epitope-binding fragments of any of the above. In certain embodiments antibodies also include affibodies, nanobodies, and unibodies. In certain embodiments particular antibodies include immunoglobulin molecules and immunologically active fragments of immunoglobulin molecules, namely, molecules that contain an antigen binding site. Immunoglobulin molecules can be of any type (for example, IgG, IgE, IgM, IgD, IgA and IgY), class (for example, IgG1, IgG2, IgG3, IgG4, IgAi and IgA2) or subclass.

As used herein, the terms “antigen-binding fragment (Fab)” refers to antibodies fragments comprising one constant and one variable domain of each of the heavy and the light chain. The variable domain contains the antigen-binding sites. Generally, an antibody comprises a fragment crystallizable region (Fc) and two antigen-binding fragments (Fab). The Fab fragments can be separated from the Fc region resulting in two Fab fragments, which is also known as F(ab′)₂ fragment or dimeric fragment antigen binding.

The term “isolated” refers to a protein (e.g., an antibody) or nucleic acid that is substantially free of other cellular material and/or chemicals. For example, when an isolated antibody is expressed by a cell from a different species, e.g., a human antibody expressed in a murine cell, and is substantially free of other proteins from the different species. A protein may be rendered substantially free of naturally associated components (or components associated with the cellular expression system used to produce the antibody) by isolation, using protein purification techniques well known in the art.

As used herein, the term “antigen” refers to a biomolecule that binds specifically to the respective antibody. An antibody from the diverse repertoire binds a specific antigenic structure by means of its variable region interaction.

As used herein, the term “epitope” refers to the portion of an antigen to which an antibody specifically binds. Thus, the term “epitope” includes any protein determinant capable of specific binding to an immunoglobulin or T-cell receptor.

A polypeptide is “immunologically reactive” with an antibody when it binds to an antibody due to antibody recognition of a specific epitope contained within the polypeptide. Immunological reactivity may be determined by antibody binding, more particularly by the kinetics of antibody binding, and/or by competition in binding using as competitor(s) a known polypeptide(s) containing an epitope against which the antibody is directed. The techniques for determining whether a polypeptide is immunologically reactive with an antibody are known in the art.

The term “sample”, as used herein, refers to any biological material obtained from a subject or patient. In one aspect, a sample can comprise blood, peritoneal fluid, CSF, saliva or urine. In other aspects, a sample can comprise whole blood, blood plasma, blood serum, B cells enriched from blood samples, and cultured cells (e.g., B cells from a subject). A sample can also include a biopsy or tissue sample including neural tissue. In still other aspects, a sample can comprise whole cells and/or a lysate of the cells.

The sample may be treated to physically or mechanically disrupt tissue or cell structure, thus releasing intracellular components into a solution which may further contain enzymes, buffers, salts, detergents and the like, which are used to prepare, using standard methods, a biological sample for analysis. Also, samples may include processed samples, such as those obtained from passing samples over or through a filtering device, or following centrifugation, or by adherence to a medium, matrix, or support.

The terms “patient” or “individual” are used interchangeably herein, and refers to a mammalian subject to be diagnosed or treated, with human patients being preferred. In some cases, the methods of the invention find use in experimental animals, in veterinary application, and in the development of animal models for disease, including, but not limited to, rodents including mice, rats, and hamsters; and primates.

The term “vector” refers to a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can direct the expression of a chosen polynucleotide. An “expression cell” is a cell that contains an expression vector.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements.

The term “diagnostic” or “diagnosed”, as used herein, means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “binding affinity”, as used herein, refers to the strength of interaction between an antigen's epitope and an antibody's antigen binding site.

The present invention relates to novel antibodies specific for the detection of Human Immunodeficiency Virus 1 (HIV-1) p24 protein. These antibodies recognize novel and non-cross-reactive epitopes of HIV-1 p24 protein and exhibit a higher degree of affinity and sensitivity when compared to other commercially-available products. Thus, the antibodies described herein can be utilized as diagnostic reagents, standards or positive controls in immunoassays for early HIV-1 detection. They can be used for detection of any of the three main HIV-1 groups (group M (main), group N (new), and group O (outlier)).

The present invention also relates to compositions and kits comprising said anti-HIV-1 antibodies for detecting the presence of HIV-1 in a sample.

### I. Anti-HIV-1 Antibodies

As used herein, the terms “homology”, “similarity” or “identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent homology/identity, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences that are compared are the same length after gaps are introduced within the sequences, as appropriate (e.g., excluding additional sequence extending beyond the sequences being compared). For sequence comparisons between two sequences, a “corresponding” CDR refers to a CDR in the same location in both sequences (e.g., CDR-H1 of each sequence).

The determination of percent identity, percent similarity, or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. When utilizing BLAST and Gapped BLAST the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In one embodiment described herein, the recombinant antibody comprises a light chain and a heavy chain. In other embodiment described herein, the recombinant antibody comprises two light chains and two heavy chains. The light chain(s) of the recombinant antibody of the present invention can comprise two domains, a variable domain (VL) and a constant domain (CL). The heavy chain(s) of the recombinant antibody of the present invention can comprise four domains, a variable domain (VH) and three constant domains (CH1, CH2 and CH3, collectively referred to as CH).

In some embodiments, the anti-HIV-1 antibody of the present invention is a monoclonal antibody. In other embodiments, the anti-HIV-1 antibody of the present invention is a recombinant antibody. In other embodiments, the anti-HIV-1 antibody is a recombinant monoclonal antibody according to the definitions of the present invention. In other embodiments, the anti-HIV-1 antibody is an isolated antibody.

In some embodiments, the anti-HIV-1 antibody is an antibody fragment. In a preferred embodiment, said antibody fragment is selected from variable fragments (Fv), single-chain Fvs (scFv), bispecific antibodies (sc(Fv)₂), single chain antibodies, single domain antibodies, Fab fragments, F(ab′)₂ fragments, Fab′ fragments, disulfide-linked Fv (dsFv), chemically conjugated Fv (ccFv), diabodies, anti-idiotypic (anti-Id) antibodies, affibodies, nanobodies, and unibodies.

In one embodiment described herein, the anti-HIV-1 antibody comprises the Fc region and the two Fab fragments. In other embodiment described herein, the anti-HIV-1 antibody is a fragment antigen binding and does not comprises the Fc region. In other embodiment described herein, the anti-HIV-1 antibody consists of one Fab fragment. In other embodiment described herein, the anti-HIV-1 antibody consists of two Fab fragments (F(ab)₂).

In one embodiment described herein, the anti-HIV-1 antibody may be of any type known by the skilled person (for example, IgG, IgE, IgM, IgD, IgA and IgY), or any class known by the skilled person (for example, IgG1, IgG2, IgG3, IgG4, IgAi and IgA2) or any known subclass.

In one embodiment described herein, the anti-HIV antibody is of the IgG type. In a preferred embodiment, the anti-HIV-antibody is of the IgG1, IgG2, IgG3 or IgG4 class. In another preferred embodiment, the anti-HIV-antibody is of the IgG1 or IgG2 class. In another preferred embodiment, the anti-HIV-antibody is of the IgG2a class.

The species of the constant region of the antibody of the present invention may be human, mouse, rabbit, rat, hamster, guinea pig, goat, sheep, horse, chicken, or a chimera of any of the foregoing species, although the species of the antibody of the present invention is not particularly limiting. In some preferred embodiments, the anti-HIV antibody of the present invention comprises a constant region of the murine IgG1 class or the murine IgG2a class.

### A. Light Chain

In some embodiments described herein, the anti-HIV-1 antibody comprises a light chain comprising complementary determining regions (CDR). Said CDRs correspond to the sequences identified according to any CDR definition approach known by the skilled person. In some preferred embodiments, the CDRs regions correspond to the sequences identified according to Kabat numbering scheme. In other preferred embodiments, the CDRs regions may correspond to the sequences identified according to other numbering methods or a combination of Kabat and other numbering methods. For example, the CDR regions may correspond to the sequences identified according to the Chothia numbering scheme.

In some embodiments described herein, the anti-HIV-1 antibody comprises a light chain comprising complementary determining regions L-CDR1, L-CDR2 and L-CDR3, each of them comprising a sequence of at least five contiguous amino acids selected from the amino acid sequence of SEQ ID NO:7, or SEQ ID NO: 8, or SEQ ID NO: 9. In some preferred embodiments, the sequence of L-CDR1 is selected from the group consisting of SEQ ID NO: 15, SEQ ID NO: 18 and SEQ ID NO: 21. In some preferred embodiments, the sequence of L-CDR2 is selected from the group consisting of SEQ ID NO: 16, SEQ ID NO: 19 and SEQ ID NO: 22. In some preferred embodiments, the sequence of L-CDR3 is selected from the group consisting of SEQ ID NO: 17, SEQ ID NO: 20 and SEQ ID NO: 23. In other preferred embodiments, the sequence of L-CDR1 is selected from the group consisting of SEQ ID NO: 15, SEQ ID NO: 18 and SEQ ID NO: 21, the sequence of L-CDR2 is selected from the group consisting of SEQ ID NO: 16, SEQ ID NO: 19 and SEQ ID NO: 22, and the sequence of L-CDR3 is selected from the group consisting of SEQ ID NO: 17, SEQ ID NO: 20 and SEQ ID NO: 23.

In another embodiment described herein, the variable region of the light chain of the anti-HIV-1 antibody of the present invention comprises the amino acid sequence of SEQ ID NO: 7, or SEQ ID NO: 8, or SEQ ID NO: 9. In yet another embodiment, the variable region of the light chain of the recombinant antibody may have about 70%, 75%, 80%, 85%, 90%, 91%, 92%, 93%, 94%, 95%, 96%, 97%, 98%, 99% or more homology to the amino acid sequence consisting of SEQ ID NO: 7, or SEQ ID NO: 8, or SEQ ID NO: 9. In some preferred embodiments, the light chain of the anti-HIV-1 antibody of the present invention comprises a sequence having about 90% homology with the amino acid sequence of SEQ ID NO: 7, or SEQ ID NO: 8, or SEQ ID NO: 9.

In another embodiment described herein, the recombinant antibody comprises a light chain comprising the amino acid sequence of SEQ ID NO: 1, SEQ ID NO: 2, or SEQ ID NO: 3. In other embodiment, the light chain of the recombinant antibody may have about 70%, 75%, 80%, 85%, 90%, 91%, 92%, 93%, 94%, 95%, 96%, 97%, 98%, 99% or more homology to the amino acid sequence consisting of SEQ ID NO: 1, SEQ ID NO: 2, or SEQ ID NO: 3. In some preferred embodiments, the light chain of the anti-HIV-1 antibody of the present invention comprises a sequence having about 90% homology with the amino acid sequence of SEQ ID NO: 1, SEQ ID NO: 2, or SEQ ID NO: 3.

### B. Heavy Chain

In some embodiments described herein, the anti-HIV-1 antibody comprises a heavy chain comprising complementary determining regions (CDR). Said CDRs correspond to the sequences identified according to any CDR definition approach known by the skilled person. In some preferred embodiments, the CDRs regions correspond to the sequences identified according to Kabat numbering scheme. In other preferred embodiments, the CDRs regions may correspond to the sequences identified according to other numbering methods or a combination of Kabat and other numbering methods. For example, the CDR regions may correspond to the sequences identified according to the Chothia numbering scheme.

In some embodiments described herein, the anti-HIV-1 antibody comprises a heavy chain comprising complementary determining regions H-CDR1, H-CDR2 and H-CDR3, each of them comprising a sequence of at least five contiguous amino acids selected from the amino acid sequence of SEQ ID NO: 10, or SEQ ID NO: 11, or SEQ ID NO: 12. In some preferred embodiments, the sequence of H-CDR1 is selected from the group consisting of SEQ ID NO: 24, SEQ ID NO: 27 and SEQ ID NO: 30. In some preferred embodiments, the sequence of H-CDR2 is selected from the group consisting of SEQ ID NO: 25, SEQ ID NO: 28 and SEQ ID NO: 31. In some preferred embodiments, the sequence of H-CDR3 is selected from the group consisting of SEQ ID NO: 26, SEQ ID NO: 29 and SEQ ID NO: 32. In some preferred embodiments, the sequence of H-CDR1 is selected from the group consisting of SEQ ID NO: 24, SEQ ID NO: 27 and SEQ ID NO: 30, the sequence of H-CDR2 is selected from the group consisting of SEQ ID NO: 25, SEQ ID NO: 28 and SEQ ID NO: 31, the sequence of H-CDR3 is selected from the group consisting of SEQ ID NO: 26, SEQ ID NO: 29 and SEQ ID NO: 32.

In another embodiment described herein, the variable region of the heavy chain of the anti-HIV-1 antibody of the present invention comprises the amino acid sequence of SEQ ID NO: 10, or SEQ ID NO: 11, or SEQ ID NO: 12. In yet another embodiment, the variable region of the heavy chain of the recombinant antibody may have about 70%, 75%, 80%, 85%, 90%, 91%, 92%, 93%, 94%, 95%, 96%, 97%, 98%, 99% or more homology to the amino acid sequence consisting of SEQ ID NO: 10, or SEQ ID NO: 11, or SEQ ID NO: 12. In some preferred embodiments, the heavy chain of the anti-HIV-1 antibody of the present invention comprises a sequence having about 90% homology with the amino acid sequence of SEQ ID NO: 10, or SEQ ID NO: 11, or SEQ ID NO: 12.

In another embodiment described herein, the recombinant antibody comprises a heavy chain comprising the amino acid sequence of SEQ ID NO: 4, SEQ ID NO: 5, or SEQ ID NO: 6. In other embodiment, the light chain of the recombinant antibody may have about 70%, 75%, 80%, 85%, 90%, 91%, 92%, 93%, 94%, 95%, 96%, 97%, 98%, 99% or more homology to the amino acid sequence consisting of SEQ ID NO: 4, SEQ ID NO: 5, or SEQ ID NO: 6. In some preferred embodiments, the heavy chain of the anti-HIV-1 antibody of the present invention comprises a sequence having about 90% homology with the amino acid sequence of SEQ ID NO: 4, SEQ ID NO: 5, or SEQ ID NO: 6.

### C. Exemplary Anti-HIV-1 Antibodies

In one embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 15, the amino acid sequence of L-CDR2 is SEQ ID NO: 16, and the amino acid sequence of L-CDR3 is SEQ ID NO: 17.

In other embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 18, the amino acid sequence of L-CDR2 is SEQ ID NO: 19, and the amino acid sequence of L-CDR3 is SEQ ID NO: 20.

In other embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 21, the amino acid sequence of L-CDR2 is SEQ ID NO: 22, and the amino acid sequence of L-CDR3 is SEQ ID NO: 23.

In one embodiment described herein, the anti-HIV-1 antibody comprises a heavy chain comprising complementary determining regions H-CDR1, H-CDR2 and H-CDR3, wherein the amino acid sequence of H-CDR1 is SEQ ID NO: 24, the amino acid sequence of H-CDR2 is SEQ ID NO: 25, and the amino acid sequence of H-CDR3 is SEQ ID NO: 26.

In other embodiment described herein, the anti-HIV-1 antibody comprises a heavy chain comprising complementary determining regions H-CDR1, H-CDR2 and H-CDR3, wherein the amino acid sequence of H-CDR1 is SEQ ID NO: 27, the amino acid sequence of H-CDR2 is SEQ ID NO: 28, and the amino acid sequence of H-CDR3 is SEQ ID NO: 29.

In other embodiment described herein, the anti-HIV-1 antibody comprises a heavy chain comprising complementary determining regions H-CDR1, H-CDR2 and H-CDR3, wherein the amino acid sequence of H-CDR1 is SEQ ID NO: 30, the amino acid sequence of H-CDR2 is SEQ ID NO: 31, and the amino acid sequence of H-CDR3 is SEQ ID NO: 32.

The anti-HIV-1 antibodies of the present invention may comprise any combination of the CDR regions of both the light and heavy chains as described herein.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 15, the amino acid sequence of L-CDR2 is SEQ ID NO: 16, and the amino acid sequence of L-CDR3 is SEQ ID NO: 17, and a heavy chain comprising complementary determining regions H-CDR1, H-CDR2 and H-CDR3, wherein the amino acid sequence of H-CDR1 is SEQ ID NO: 24, the amino acid sequence of H-CDR2 is SEQ ID NO: 25, and the amino acid sequence of H-CDR3 is SEQ ID NO: 26.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 15, the amino acid sequence of L-CDR2 is SEQ ID NO: 16, and the amino acid sequence of L-CDR3 is SEQ ID NO: 17, and a heavy chain comprising complementary determining regions H-CDR1, H-CDR2 and H-CDR3, wherein the amino acid sequence of H-CDR1 is SEQ ID NO: 27, the amino acid sequence of H-CDR2 is SEQ ID NO: 28, and the amino acid sequence of H-CDR3 is SEQ ID NO: 29.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 15, the amino acid sequence of L-CDR2 is SEQ ID NO: 16, and the amino acid sequence of L-CDR3 is SEQ ID NO: 17, and a heavy chain comprising complementary determining regions H-CDR1, H-CDR2 and H-CDR3, wherein the amino acid sequence of H-CDR1 is SEQ ID NO: 30, the amino acid sequence of H-CDR2 is SEQ ID NO: 31, and the amino acid sequence of H-CDR3 is SEQ ID NO: 32.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 18, the amino acid sequence of L-CDR2 is SEQ ID NO: 19, and the amino acid sequence of L-CDR3 is SEQ ID NO: 20, and a heavy chain comprising complementary determining regions H-CDR1, H-CDR2 and H-CDR3, wherein the amino acid sequence of H-CDR1 is SEQ ID NO: 24, the amino acid sequence of H-CDR2 is SEQ ID NO: 25, and the amino acid sequence of H-CDR3 is SEQ ID NO: 26.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 18, the amino acid sequence of L-CDR2 is SEQ ID NO: 19, and the amino acid sequence of L-CDR3 is SEQ ID NO: 20, and a heavy chain comprising complementary determining regions H-CDR1, H-CDR2 and H-CDR3, wherein the amino acid sequence of H-CDR1 is SEQ ID NO: 27, the amino acid sequence of H-CDR2 is SEQ ID NO: 28, and the amino acid sequence of H-CDR3 is SEQ ID NO: 29.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 18, the amino acid sequence of L-CDR2 is SEQ ID NO: 19, and the amino acid sequence of L-CDR3 is SEQ ID NO: 20, and a heavy chain comprising complementary determining regions H-CDR1, H-CDR2 and H-CDR3, wherein the amino acid sequence of H-CDR1 is SEQ ID NO: 30, the amino acid sequence of H-CDR2 is SEQ ID NO: 31, and the amino acid sequence of H-CDR3 is SEQ ID NO: 32.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 21, the amino acid sequence of L-CDR2 is SEQ ID NO: 22, and the amino acid sequence of L-CDR3 is SEQ ID NO: 23, and a heavy chain comprising complementary determining regions H-CDR1, H-CDR2 and H-CDR3, wherein the amino acid sequence of H-CDR1 is SEQ ID NO: 24, the amino acid sequence of H-CDR2 is SEQ ID NO: 25, and the amino acid sequence of H-CDR3 is SEQ ID NO: 26.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 21, the amino acid sequence of L-CDR2 is SEQ ID NO: 22, and the amino acid sequence of L-CDR3 is SEQ ID NO: 23, and a heavy chain comprising complementary determining regions H-CDR1, H-CDR2 and H-CDR3, wherein the amino acid sequence of H-CDR1 is SEQ ID NO: 27, the amino acid sequence of H-CDR2 is SEQ ID NO: 28, and the amino acid sequence of H-CDR3 is SEQ ID NO: 29.

In a preferred embodiment described herein, the anti-HIV-1 antibody comprises a light chain comprising complementary determining regions L-CDR1, L-CDR2 and L-CDR3, wherein the amino acid sequence of L-CDR1 is SEQ ID NO: 21, the amino acid sequence of L-CDR2 is SEQ ID NO: 22, and the amino acid sequence of L-CDR3 is SEQ ID NO: 23, and a heavy chain comprising complementary determining regions H-CDR1, H-CDR2 and H-CDR3, wherein the amino acid sequence of H-CDR1 is SEQ ID NO: 30, the amino acid sequence of H-CDR2 is SEQ ID NO: 31, and the amino acid sequence of H-CDR3 is SEQ ID NO: 32.

In one embodiment described herein, the anti-HIV-1 antibody comprises a light chain comprising the amino acid sequence selected from the group consisting of SEQ ID NO: 7, SEQ ID NO: 8, or SEQ ID NO: 9 and a heavy chain comprising the amino acid sequence selected from the group consisting of SEQ ID NO: 10, SEQ ID NO: 11, or SEQ ID NO: 12.

In a preferred embodiment, the anti-HIV-1 antibody comprises a light chain comprising the amino acid sequence of SEQ ID NO: 7, and a heavy chain comprising the amino acid sequence of SEQ ID NO: 10.

In a preferred embodiment, the anti-HIV-1 antibody comprises a light chain comprising the amino acid sequence of SEQ ID NO: 7, and a heavy chain comprising the amino acid sequence of SEQ ID NO: 11.

In a preferred embodiment, the anti-HIV-1 antibody comprises a light chain comprising the amino acid sequence of SEQ ID NO: 7, and a heavy chain comprising the amino acid sequence of SEQ ID NO: 12.

In a preferred embodiment, the anti-HIV-1 antibody comprises a light chain comprising the amino acid sequence of SEQ ID NO: 8, and a heavy chain comprising the amino acid sequence of SEQ ID NO: 10.

In a preferred embodiment, the anti-HIV-1 antibody comprises a light chain comprising the amino acid sequence of SEQ ID NO: 8, and a heavy chain comprising the amino acid sequence of SEQ ID NO: 11.

In a preferred embodiment, the anti-HIV-1 antibody comprises a light chain comprising the amino acid sequence of SEQ ID NO: 8, and a heavy chain comprising the amino acid sequence of SEQ ID NO: 12.

In a preferred embodiment, the anti-HIV-1 antibody comprises a light chain comprising the amino acid sequence of SEQ ID NO: 9, and a heavy chain comprising the amino acid sequence of SEQ ID NO: 10.

In a preferred embodiment, the anti-HIV-1 antibody comprises a light chain comprising the amino acid sequence of SEQ ID NO: 9, and a heavy chain comprising the amino acid sequence of SEQ ID NO: 11.

In a preferred embodiment, the anti-HIV-1 antibody comprises a light chain comprising the amino acid sequence of SEQ ID NO: 9, and a heavy chain comprising the amino acid sequence of SEQ ID NO: 12.

In other preferred embodiments, the light chain of the anti-HIV-1 antibody may have about 70%, 75%, 80%, 85%, 90%, 91%, 92%, 93%, 94%, 95%, 96%, 97%, 98%, 99% or more homology to the amino acid sequence consisting of SEQ ID NO: 7, SEQ ID NO: 8, or SEQ ID NO: 9 and the heavy chain of the anti-HIV-1 antibody may have about 70%, 75%, 80%, 85%, 90%, 91%, 92%, 93%, 94%, 95%, 96%, 97%, 98%, 99% or more homology to the amino acid sequence consisting of SEQ ID NO: 10, SEQ ID NO: 11, or SEQ ID NO: 12.

In one embodiment described herein, the anti-HIV-1 antibody comprises a light chain comprising the amino acid sequence selected from the group consisting of SEQ ID NO: 1, SEQ ID NO: 2, or SEQ ID NO: 3 and a heavy chain comprising the amino acid sequence selected from the group consisting of SEQ ID NO: 4, SEQ ID NO: 5, or SEQ ID NO: 6.

In a preferred embodiment, the anti-HIV-1 antibody comprises a light chain comprising the amino acid sequence of SEQ ID NO: 1, and a heavy chain comprising the amino acid sequence of SEQ ID NO: 4.

In a preferred embodiment, the anti-HIV-1 antibody comprises a light chain comprising the amino acid sequence of SEQ ID NO: 1, and a heavy chain comprising the amino acid sequence of SEQ ID NO: 5.

In a preferred embodiment, the anti-HIV-1 antibody comprises a light chain comprising the amino acid sequence of SEQ ID NO: 1, and a heavy chain comprising the amino acid sequence of SEQ ID NO: 6.

In a preferred embodiment, the anti-HIV-1 antibody comprises a light chain comprising the amino acid sequence of SEQ ID NO: 2, and a heavy chain comprising the amino acid sequence of SEQ ID NO: 4.

In a preferred embodiment, the anti-HIV-1 antibody comprises a light chain comprising the amino acid sequence of SEQ ID NO: 2, and a heavy chain comprising the amino acid sequence of SEQ ID NO: 5.

In a preferred embodiment, the anti-HIV-1 antibody comprises a light chain comprising the amino acid sequence of SEQ ID NO: 2, and a heavy chain comprising the amino acid sequence of SEQ ID NO: 6.

In a preferred embodiment, the anti-HIV-1 antibody comprises a light chain comprising the amino acid sequence of SEQ ID NO: 3, and a heavy chain comprising the amino acid sequence of SEQ ID NO: 4.

In a preferred embodiment, the anti-HIV-1 antibody comprises a light chain comprising the amino acid sequence of SEQ ID NO: 3, and a heavy chain comprising the amino acid sequence of SEQ ID NO: 5.

In a preferred embodiment, the anti-HIV-1 antibody comprises a light chain comprising the amino acid sequence of SEQ ID NO: 3, and a heavy chain comprising the amino acid sequence of SEQ ID NO: 6.

In other preferred embodiments, the light chain of the anti-HIV-1 antibody may have about 70%, 75%, 80%, 85%, 90%, 91%, 92%, 93%, 94%, 95%, 96%, 97%, 98%, 99% or more homology to the amino acid sequence consisting of SEQ ID NO: 1, SEQ ID NO: 2, or SEQ ID NO: 3, and the heavy chain of the anti-HIV-1 antibody may have about 70%, 75%, 80%, 85%, 90%, 91%, 92%, 93%, 94%, 95%, 96%, 97%, 98%, 99% or more homology to the amino acid sequence consisting of SEQ ID NO: 4, SEQ ID NO: 5, or SEQ ID NO: 6.

In some preferred embodiments, the anti-HIV-1 antibodies of the present invention specifically bind to HIV-1 p24 protein. In some embodiments, the anti-HIV-1 antibodies of the present invention bind to an epitope of HIV-1 p24 protein. In some preferred embodiments, the anti-HIV-1 antibodies of the present invention bind to a linear epitope of HIV-1 p24 protein. In some preferred embodiments, the anti-HIV-1 antibodies of the present invention bind to a linear epitope comprising at least five contiguous amino acids selected from the amino acid sequence of HIV-1 p24 protein (SEQ ID NO: 35) or a sequence having at least 90% homology with said sequence. In other embodiments, the amino acid sequence of HIV-1 p24 protein is set forth in SEQ ID NO: 36.

In other preferred embodiments, the anti-HIV-1 antibodies of the present invention bind to an epitope of HIV-1 p24 protein characterized in that said epitope comprises the amino acid sequence of SEQ ID NO: 33.

In more preferred embodiments, the anti-HIV-1 antibodies of the present invention bind to an epitope of HIV-1 p24 protein characterized in that said epitope comprises an amino acid sequence selected from the group consisting of SEQ ID NO: 33, and wherein said antibody comprises a light chain comprising complementary determining regions L-CDR1, L-CDR2 and L-CDR3, wherein the amino acid sequence of L-CDR1 is selected from the group consisting of SEQ ID NO: 15, SEQ ID NO: 18 and SEQ ID NO: 21, the amino acid sequence of L-CDR2 is selected from the group consisting of SEQ ID NO: 16, SEQ ID NO: 19 and SEQ ID NO: 22, and the amino acid sequence of L-CDR3 is selected from the group consisting of SEQ ID NO: 17, SEQ ID NO: 20 and SEQ ID NO: 23, and further comprise a heavy chain comprising complementary determining regions H-CDR1, H-CDR2 and H-CDR3, wherein the amino acid sequence of H-CDR 1 is selected from the group consisting of SEQ ID NO: 24, SEQ ID NO: 27 and SEQ ID NO: 30, the amino acid sequence of H-CDR2 is selected from the group consisting of SEQ ID NO: 25, SEQ ID NO: 28 and SEQ ID NO: 31, and the amino acid sequence of H-CDR3 is selected from the group consisting of SEQ ID NO: 26, SEQ ID NO: 29 and SEQ ID NO: 32.

In some preferred embodiments, the anti-HIV-1 antibodies of the present invention bind to an epitope of HIV-1 p24 protein characterized in that said epitope comprises the amino acid sequence of SEQ ID NO: 33, and wherein said antibody comprises a light chain comprising complementary determining regions L-CDR1, L-CDR2 and L-CDR3, wherein the amino acid sequence of L-CDR1 is SEQ ID NO: 18, the amino acid sequence of L-CDR2 is SEQ ID NO: 19, and the amino acid sequence of L-CDR3 is SEQ ID NO: 20, and further comprise a heavy chain comprising complementary determining regions H-CDR1, H-CDR2 and H-CDR3, wherein the amino acid sequence of H-CDR1 is SEQ ID NO: 27, the amino acid sequence of H-CDR2 is SEQ ID NO: 28, and the amino acid sequence of H-CDR3 is SEQ ID NO: 29.

In some embodiments, the anti-HIV-1 antibody of the present invention is bound to a solid support.

### D. Affinity Tags

An anti-HIV-1 antibody according to the present invention may optionally include an affinity tag. Affinity tags are useful for purification. Exemplary affinity tags include polyhistidine, Glutathione S-transferase (GST), chitin binding protein, maltose binding protein (MBP), streptavidin binding peptide (Strep-tag), isopeptide bond forming, FLAG-tag, V5-tag, Myc-tag, HA-tag, NE-tag, AviTag, Calmodulin-tag, polyglutamate, S-tag, SBP-tag, Softag 1, Softag 3, TC tag, VSV-tag, Xpress tag, Isopeptag, SpyTag, SnoopTag, biotin carboxyl carrier protein, green fluorescent protein-tag, HaloTag, Nus-tag, and thioredoxin-tag, although the choice of affinity tag is not particularly limiting. A anti-HIV-1 antibody may nevertheless lack an affinity tag, for example, if the affinity tag is removed after use or if the antibody is purified using a strategy that does not require an affinity tag. An exemplary affinity tag is polyhistidine, which typically includes an amino acid sequence comprising between 4 and 10 consecutive histidines.

The anti-HIV-1 antibodies of the present invention may optionally include an affinity tag and may optionally be purified using said affinity tag. Several methods of purification of anti-HIV-1 antibodies are available in the state of the art and the skilled person is well aware of them. Exemplary methods of purification for anti-HIV-1 antibodies, comprising or not an affinity tags, are immobilized metal affinity chromatography (IMAC), Protein A/G affinity, exchange chromatography (IEX or IEC), hydrophobic interaction chromatography (HIC) and/or additional use of tags and affinity chromatography techniques beyond IMAC or Protein A/G. The purification method and tags utilized should not be considered limiting.

### II. Nucleic Acids, Cloning Cells, and Expression Cells

The present invention also relates to nucleic acids comprising a nucleotide sequence encoding the anti-HIV-1 antibodies described herein. The nucleic acid may be an isolated nucleic acid. The nucleic acid may be DNA or RNA. DNA comprising a nucleotide sequence encoding an anti-HIV-1 antibody described herein typically comprises a promoter that is operably-linked to the nucleotide sequence. The promoter is preferably capable of driving constitutive or inducible expression of the nucleotide sequence in an expression cell of interest. Said nucleic acid may also comprise a selectable marker useful to select the cell containing said nucleic acid of interest. Useful selectable markers are well known by the skilled person. The precise nucleotide sequence of the nucleic acid is not particularly limiting so long as the nucleotide sequence encodes an anti-HIV-1 antibody described herein. Codons may be selected, for example, to match the codon bias of an expression cell of interest (e.g., a mammalian cell such as a human cell) and/or for convenience during cloning. DNA may be a plasmid, for example, which may comprise an origin of replication (e.g., for replication of the plasmid in a prokaryotic cell).

In one embodiment described herein, the nucleic acid comprises a nucleotide sequence encoding the anti-HIV-1 antibody of the present invention, a promoter operably linked to the nucleotide sequence and a selectable marker.

In some preferred embodiments, the nucleic acid comprises the nucleotide sequence selected from the group consisting of SEQ ID NO: 37, SEQ ID NO: 38, SEQ ID NO: 39, SEQ ID NO: 40, SEQ ID NO: 41 and SEQ ID NO: 42. In more preferred embodiments, the nucleic acid of the light chain of the anti-HIV-1 antibody of the present invention comprises the nucleotide sequence selected from the group consisting of SEQ ID NO: 37, SEQ ID NO: 39, and SEQ ID NO: 41, and the nucleic acid of the heavy chain of the anti-HIV-1 antibody of the present invention comprises the nucleotide sequence selected from the group consisting of SEQ ID NO: 38, SEQ ID NO: 40 and SEQ ID NO: 42.

In some embodiments, the nucleic acid of the light and heavy chains of the anti-HIV-1 antibody of the present invention comprises respectively the nucleotide sequence of SEQ ID NO: 37 and the nucleotide sequence of SEQ ID NO: 38. In other embodiments, the nucleic acid of the light and heavy chains of the anti-HIV-1 antibody of the present invention comprises respectively the nucleotide sequence of SEQ ID NO: 39 and the nucleotide sequence of SEQ ID NO: 40. In other embodiments, the nucleic acid of the light and heavy chains of the anti-HIV-1 antibody of the present invention comprises respectively the nucleotide sequence of SEQ ID NO: 41 and the nucleotide sequence of SEQ ID NO: 42.

Various aspects of the present invention also relate to a cell comprising a nucleic acid comprising a nucleotide sequence that encodes an anti-HIV-1 antibody as described herein. The cell may be an expression cell or a cloning cell. Nucleic acids are typically cloned in E. coli, although other cloning cells may be used.

If the cell is an expression cell, the nucleic acid is optionally a nucleic acid of a chromosome, i.e., wherein the nucleotide sequence is integrated into the chromosome, although the nucleic acid may be present in an expression cell, for example, as extrachromosomal DNA or vectors, such as plasmids, cosmids, phages, etc. The format of the vector should not be considered limiting.

In one embodiment described herein, the cell is typically an expression cell. The nature of the expression cell is not particularly limiting. Mammalian expression cells may allow for favorable folding, post-translational modifications, and/or secretion of a recombinant antibody or oligomeric recombinant antibody, although other eukaryotic cells or prokaryotic cells may be used as expression cells. Exemplary expression cells include CHO cell lines, such as TunaCHO or ExpiCHO, Expi293, BHK, NSO, Sp2/0, COS, C127, HEK, HT-1080, PER.C6, HeLa, and Jurkat cells. The cell may also be selected for integration of a vector, more preferably for integration of a plasmid DNA.

The anti-HIV-1 antibodies of the present invention can be produced by appropriate transfection strategy of the nucleic acids comprising a nucleotide sequence that encodes the anti-HIV-1 antibodies into mammalian cells. The skilled person is aware of the different techniques available for transfection of nucleic acids into the cell line of choice (lipofection, electroporation, etc). Thus, the choice of the mammalian cell line and transfection strategy should not be considered limiting. The cell line could be further selected for integration of the plasmid DNA.

In one preferred embodiment described herein, the cell comprises the anti-HIV-1 antibody of the present invention.

### III. Compositions and Kits

Various aspects of the present invention relate to compositions comprising an anti-HIV-1 antibody as described herein.

In one embodiment described herein, the composition comprises the anti-HIV-1 antibody of the present invention and a solid support.

In other embodiment, the composition comprises the anti-HIV-1 antibody of the present invention and a solid support, wherein the anti-HIV-1 antibody is covalently or non-covalently bound to the solid support. The term “non-covalently bound,” as used herein, refers to specific binding such as between an antibody and its antigen, a ligand and its receptor, or an enzyme and its substrate, exemplified, for example, by the interaction between streptavidin binding protein and streptavidin or an antibody and its antigen.

In other embodiment, the composition comprises the anti-HIV-1 antibody of the present invention and a solid support, wherein the anti-HIV-1 antibody is directly or indirectly bound to a solid support. The term “direct” binding, as used herein, refers to the direct conjugation of a molecule to a solid support, e.g., a gold-thiol interaction that binds a cysteine thiol of an anti-HIV-1 antibody to a gold surface. The term “indirect” binding, as used herein, includes the specific binding of an anti-HIV-1 antibody to another molecule that is directly bound to a solid support, e.g., an anti-HIV-1 antibody may bind an antibody that is directly bound to a solid support thereby indirectly binding the anti-HIV-1 antibody to the solid support. The term “indirect” binding is independent of the number of molecules between the anti-HIV-1 antibody and the solid support so long as (a) each interaction between the daisy chain of molecules is a specific or covalent interaction and (b) a terminal molecule of the daisy chain is directly bound to the solid support.

A solid support may comprise a particle, a bead, a membrane, a surface, a polypeptide chip, a microtiter plate, or the solid-phase of a chromatography column. Preferably, the solid support may be a latex bead.

A composition may comprise a plurality of beads or particles, wherein each bead or particle of the plurality of beads or particles are directly or indirectly bound to at least one anti-HIV-1 antibody as described herein. A composition may comprise a plurality of beads or particles, wherein each bead or particle of the plurality of beads or particles are covalently or non-covalently bound to at least one anti-HIV-1 antibody as described herein.

Various aspects of the embodiments relate to a kit for detecting the presence of HIV-1 in a sample, said kit comprising at least one anti-HIV-1 antibody and a solid support or composition as described herein. In some embodiments, the at least one antibody is covalently or non-covalently bound to a solid support.

The anti-HIV-1 antibodies, compositions and kits described herewith can be for use, for example, in assays for detecting the presence of HIV-1 in a sample or for measuring the concentration of HIV-1 in a sample, but they are not limited to said assays. The anti-HIV-1 antibodies, compositions and kits of the present invention can also be used for detection of HIV-1 only or in combination with other antibodies for detection of other pathogens, such as multiplex assays and methods.

In some preferred embodiments, the anti-HIV-1 antibodies of the present invention are used in methods and assays in which other RNA viruses are also detected. In other embodiments, said anti-HIV-1 antibodies and other anti-HIV-2 antibodies are used in methods and assays for simultaneous detection of HIV-1 and HIV-2 in a sample. In more preferred embodiments, said anti-HIV-1 antibodies and other anti-HIV-2 antibodies are used in methods and assays for specific detection of HIV-1 p24 protein and HIV-2 p26 protein in a sample.

It is also contemplated within the scope of the present invention the use of more than one anti-HIV-1 antibodies as described herein in methods and assays for detection of HIV-1 in a sample.

Hereinafter, the present invention is described in more detail with reference to illustrative examples, which does not constitute a limitation of the present invention.

### EXEMPLIFICATION

### Example 1: Preferred Anti-HIV-Antibodies and Stable Cell Line Production

Specific combination of the light and heavy chains of the present invention resulted in preferred antibodies, as disclosed herein:

TABLE 1
Preferred combination of light and heavy
chains of anti-HIV-1 antibodies

| Antibody | Light chain | Heavy Chain |
|---|---|---|
| #A | SEQ ID NO: 1 | SEQ ID NO: 4 |
| #B | SEQ ID NO: 2 | SEQ ID NO: 5 |
| #D | SEQ ID NO: 3 | SEQ ID NO: 6 |

The variable and constant regions for each antibody were cloned into a bicistronic vector and expressed in Chinese hamster ovary (CHO) cells. Manufacturing characteristics for each antibody were evaluated based on the capability to generate stable cell line clones for each one, as well as the reproducible expression and purification of functional antibodies.

### Pool Development

Transfection: Expression of three constructs containing the nucleotides sequences for antibodies #A, #B and #D (SEQ ID NOs: 37 to 42) were generated in a bicistronic expression vector containing the heavy chain and light chain of each antibody. To express the antibodies, CHO cells were electroporated with 200 μg of DNA to create stable cell lines. Twenty-four hours later, the transfected cells were counted and placed under selection media for stable integration of the protein genes.

Pool Generation: The transfected cells were seeded into selection media at a cell density of 0.5×10⁶ cells/mL in a 250 mL shaker flask with 50 mL working volume and incubated at 37° C. with 5% CO2. During the selection process the cells were spun down and resuspended in fresh selection media every 2-3 days until the pool recovered its growth rate and viability. The cell culture was monitored for growth, via viable cell density (VCD) and percent viability, and titer.

Production Pool: One liter production runs were performed from stable pools to evaluate the VCD, titer, and viability. The cells were scaled up in production media in 3 L shake flasks (1 L working volume). The conditioned media supernatant harvested from each stable pool production run was clarified by centrifuge spinning and protein was purified by affinity purification using a Protein A column (Tables 2-4).

Cell Line Banking: Cells were grown to 2.5×10⁶ cells per mL. At the time of harvest for cell banking, the viability was above 95%. The cells were then centrifuged, and the cell pellet was resuspended in CHO complete media with 7.5% dimethylsulfoxide (DMSO) (Sigma-Aldrich, D1435) to a cell count of 15×10⁶ cells per mL per vial. Five total vials of each of the pools were produced and cryopreserved for storage in liquid nitrogen.

TABLE 2
Production and purification for antibody #A pools 1 and 2.

| | Step of stable pool | | | | |
|---|---|---|---|---|---|
| Antibody #A | evaluation | Pool 1 | | Pool 2 | |
| Cell culturing | Duration of Production Run | 17 | days | 15 | days |
| | Expression titer at harvest | 9 | mg/L | 2.63 | mg/L |
| Purification | Volume of load onto | 1 | L | 1 | L |
| | purification resin | | | | |
| | Yield obtained from | 8.45 | mg | 3.85 | mg |
| | purification | | | | |
| | Purification titer | 8.45 | mg/L | 4.13 | mg/L |

TABLE 3
Production and purification process for antibody #B pools 1 and 2.

| | Step of stable pool | | | | |
|---|---|---|---|---|---|
| Antibody #B | evaluation | Pool 1 | | Pool 2 | |
| Cell culturing | Duration of Production Run | 18 | days | 15 | days |
| | Expression titer at harvest | 209 | mg/L | 1.9 | mg/L |
| Purification | Volume of load onto | 1 | L | 1 | L |
| | purification resin | | | | |
| | Yield obtained from | 97.74 | mg | 0.88 | mg |
| | purification | | | | |
| | Purification titer | 97.74 | mg/L | 1.16 | mg/L |

TABLE 4
Production and purification process for antibody #D pools 1 and 2.

| | Step of stable pool | | | | |
|---|---|---|---|---|---|
| Antibody #D | evaluation | Pool 1 | | Pool 2 | |
| Cell culturing | Duration of Production Run | 18 | days | 17 | days |
| | Expression titer at harvest | 34.4 | mg/L | 4.15 | mg/L |
| Purification | Volume of load onto | 1 | L | 1 | L |
| | purification resin | | | | |
| | Yield obtained from | 84.45 | mg | 5.70 | mg |
| | purification | | | | |
| | Purification titer | 84.45 | mg/L | 6.71 | mg/L |

### Stable Antibody Generation

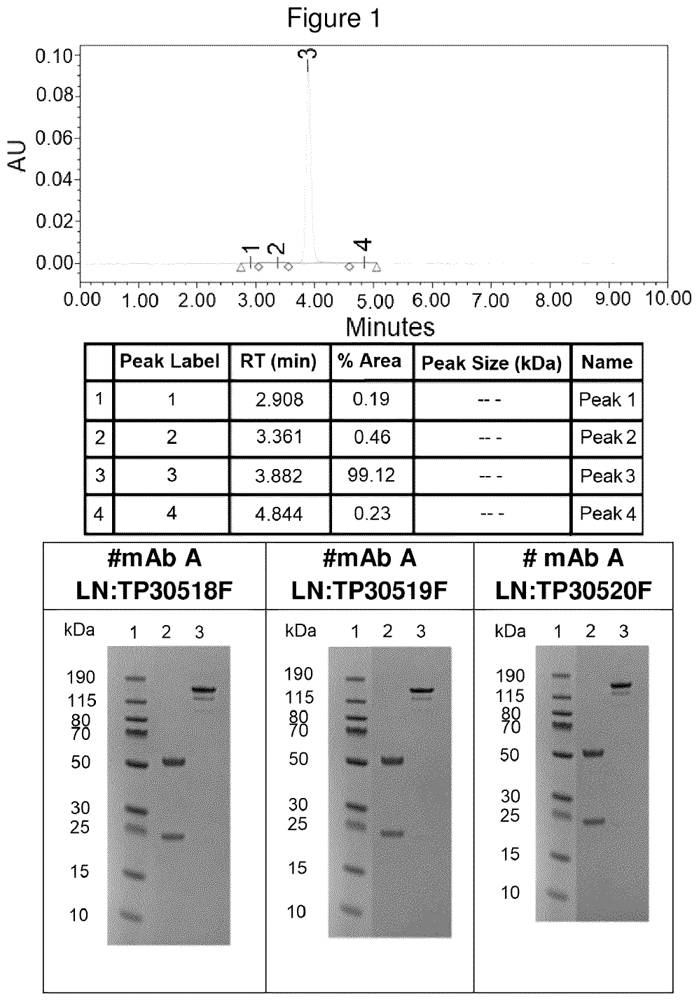

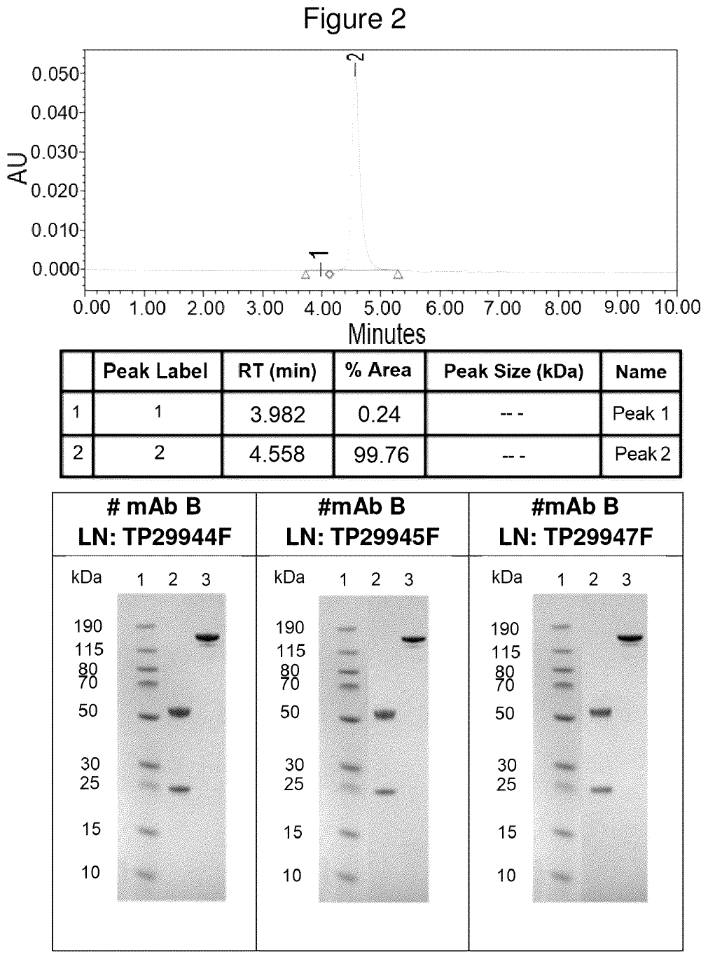

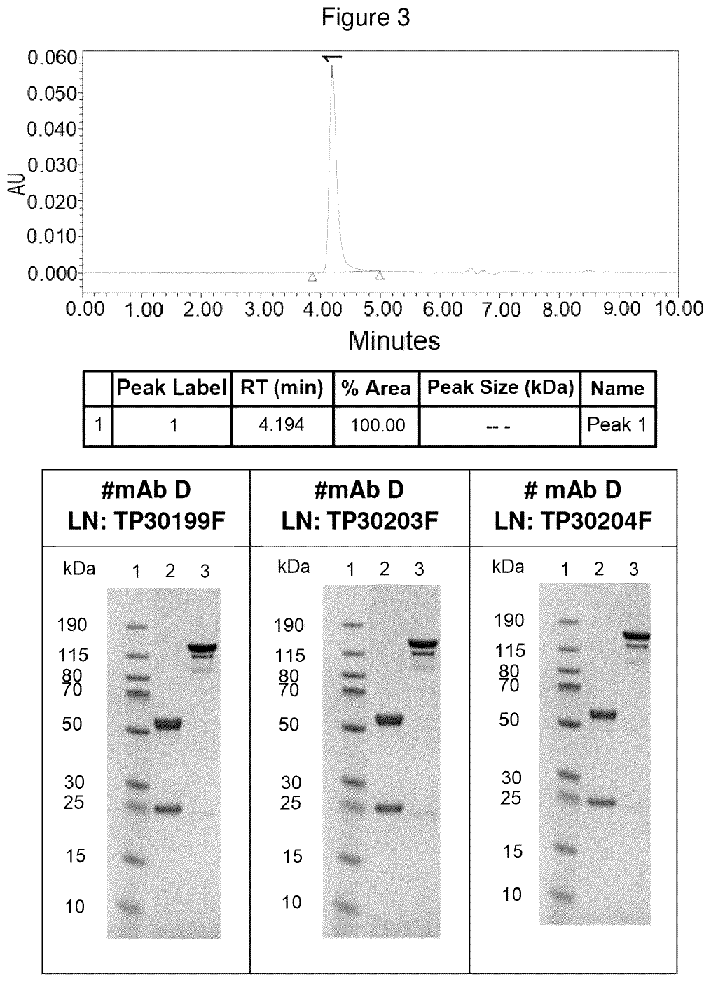

Starting from the best banked pool cell line for antibodies #A, #B and #D, stable clones were obtained via single cell cloning. The best clones for each antibody were selected based on expression level and the bioanalytical characterization of purified material for antibodies #A, #B and #D from the production runs. The bioanalytical characterization included SE-UPLC and SDS-PAGE (FIGS. 1-3).

### Example 2: Antibody Modelling and Evaluation

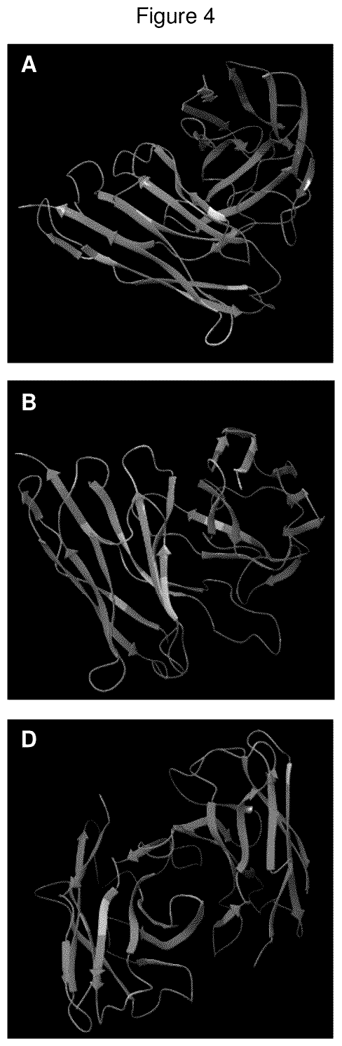

A three-dimensional structure model of antibodies #A, #B and #D was built by antibody homology using the computational and modelling software Bioiluminate (Schrodinger), version 3.5. Briefly, the amino acid sequences for the VH and VL regions of antibodies #A, #B and #D were loaded to Bioiluminate. Framework regions and CDRs were identified through searching the antibody structures in the Protein Data Bank (PDB) and selecting a PDB template based on high sequence similarity and structural fitness (Table 5). The predicted CDR sequences for each of the antibodies of the present invention are shown in Table 5 and the PDB predicted structure are shown in FIGS. 4A, 4B and 4D respectively for antibody #A, #B and #D. For antibody #A the PDB structure code 2XKN was used in the homology query, while codes 5OPY and 1F3D were used respectively for antibodies B # and #D.

Antibodies #A and #B resulted of the IgG1k isotype while antibody #D resulted of the IgG2ak isotype.

TABLE 5
Chothia CDR sequences of the light and heavy
chains of antibodies #A, #B and #D derived
according to the Abysis database website.

| Antibody | CDR | Light chain | Heavy Chain |
|---|---|---|---|
| #A | CDR1 | RASQDISNYLH | GFTFSSY |
| | CDR2 | YTSRLHS | TSGGN |
| | CDR3 | QQGNSFPWT | EVLSVPFAY |
| #B | CDR1 | RASQSISDNLH | GFAFSSY |
| | CDR2 | YSSQSIS | TSGVGN |
| | CDR3 | QQSNSWPFT | PPSYFGSSYDAMDY |
| #D | CDR1 | RSSQSLVNSDGNTFLQ | GYAFTSY |
| | CDR2 | KVSNRFS | DPYNGG |
| | CDR3 | SQSTHVPWT | PRWLPAGDY |

Analysis of nucleotide sequences of the three antibodies show that all generated heavy (VH) and light chains (VL) have unique complementary determining regions (CDR) when queried against IgBLAST, an algorithm developed by the National Center for Biotechnology Information (NCBI) to facilitate analysis of immunoglobulin variable domain sequences against the ImMunoGeneTics Database (IMGT) database (Lefranc M-P. Lefranc G. IMGT® and 30 years of Immunoinformatics Insight in Antibody V and C Domain Structure and Function. In Jefferis R; Strohl W. R., Kato K. Antibodies 2019, vol. 8(29); 1-21).

### Example 3: Epitope Mapping mAb D

The sequence of HIV-p24 was elongated by neutral GSGSGSG linkers at the C- and N-terminus to avoid truncated peptides. The elongated antigen sequence was translated into linear 15 amino acid peptides with a peptide-peptide overlap of 14 amino acids. The resulting HIV-p24 peptide microarrays contained 232 different linear peptides printed in duplicate (464 spots) and were framed by additional HA (YPYDVPDYAG, 38 spots) and c-Myc (EQKLISEEDL, 38 spots) control peptides.

Washing Buffer: PBS, pH 7.4 with 0.05% Tween 20; washing for 3×10 sec after each incubation step

Blocking Buffer: Rockland blocking buffer MB-070 (30 min before the first assay)

Incubation Buffer: Washing buffer with 10% blocking buffer

Assay Conditions: Antibody concentrations of 1 μg/ml, 10 μg/ml and 100 μg/ml in incubation buffer; incubation for 16 h at 4° C.; shaking at 140 rpm

Secondary Antibody: Goat anti-mouse IgG (H+L) DyLight680 (0.2 μg/ml); 45 min staining in incubation buffer at RTControl Antibody: Mouse monoclonal anti-HA (12CA5) DyLight800 (0.5 μg/ml); 45 min staining in incubation buffer at RT

Scanner: LI-COR Odyssey Imaging System; scanning offset 0.65 mm, resolution 21 μm, scanning intensities of 7/7 (red=680 nm/green=800 nm)

Pre-staining of a HIV-p24 peptide microarray was done with the secondary goat anti-mouse IgG (H+L) DyLight680 antibody in incubation buffer to investigate background interactions with the antigenderived peptides that could interfere with the main assays. Subsequent incubation of other HIV-p24 peptide microarray copies with monoclonal antibody D at concentrations of 1 μg/ml, 10 μg/ml and 100 μg/ml in incubation buffer was followed by staining with the secondary and control antibodies as well as read-out at scanning intensities of 7/7 (red/green). The additional HA peptides framing the peptide microarrays were subsequently stained as internal quality control to confirm the assay quality and the peptide microarray integrity.

Epitope mapping against HIV-p24 for mAb D, and a subsequent epitope substitution scan highlighted a conserved seven amino acid core motif PIAPGQM (SEQ ID NO:33).

### Example 4: Epitope Binning Studies

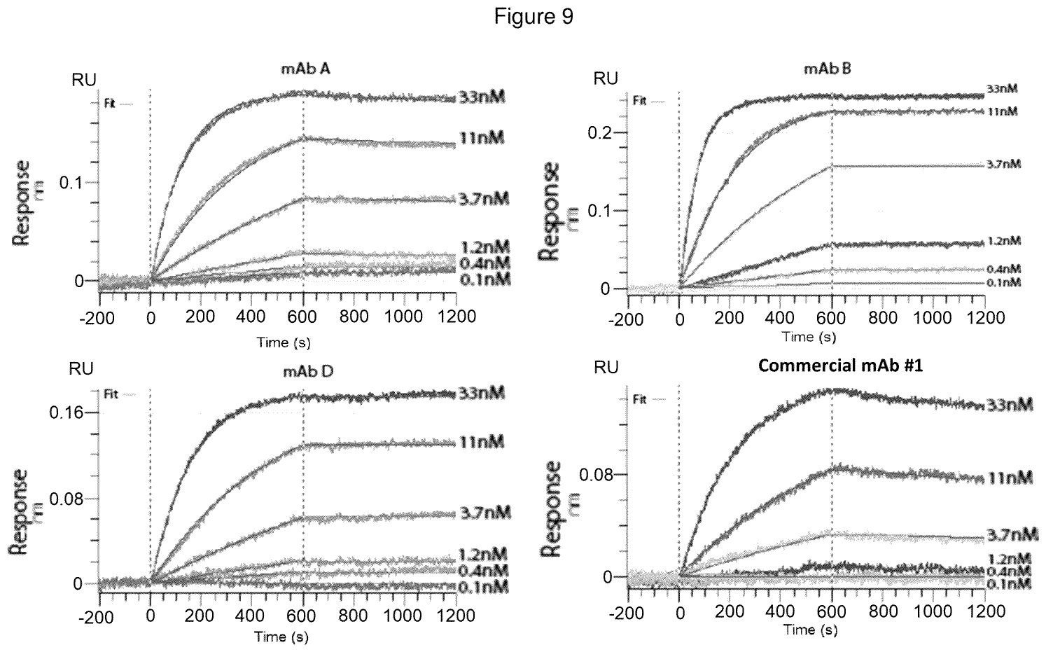

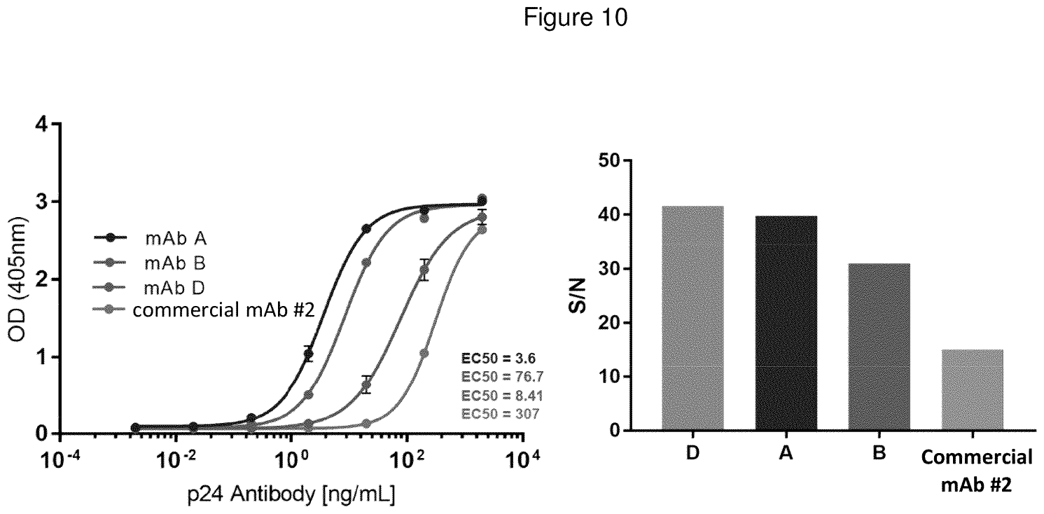

The molecular docking and western blot evaluation of antibodies #A, #B and #D suggested that these antibodies recognize linear epitopes at regions 1, 4 and 7 respectively, of the HIV-1 p24 protein. To further confirm these observations, a tandem epitope binning assay was performed using Biolayer Interferometry (BLI). A yeast-derived version of HIV-1 p24 antigen was biotinylated (bt-p24) and loaded onto Streptavidin (SA) biosensors for 300 seconds. Loaded sensors were dipped into saturating antibody (100 μg/mL) for 600 seconds followed by competing antibody (25 μg/mL) for 300 seconds. The results show that when antibody #A binds to HIV-1 p24, antibodies #B and #D add an increase in the BLI signal response, demonstrating that antibodies #B and #D bind to distinct epitopes when compared to antibody A. Similarly, if antibodies #A or #D are used as saturating antibodies, the remainder antibodies do not show competition for the same epitope (FIG. 9-10).

Table 6 summarizes epitope binning data for antibodies #A, #B and #D. Briefly, the BLI signals for competing and saturating antibodies were normalized against the buffer. The threshold for determination of antibody blocking or binding was set at 0.02 so that self-blocking pairs could be recognized in the diagonal of the matrix (grey refers to binding and bold font to self-blocking). PEARSON correlation coefficients were calculated against the first antibody #A using PEARSON function in Microsoft Excel (Liao-Chan S., et al., Monoclonal Antibody Binding-site Diversity Assessment with a Cell-based Clustering Assay. Journal of Immunological Methods 2014, vol. 405; 1-14). Three distinct bins were identified for antibodies #A, #B and #D. No antibody blocking was observed.

### Example 5: Affinity Evaluation of Anti-HIV-1 Antibodies #a, #B and #D

To investigate in more detail the interaction between antibodies #A, #B and #D and the HIV-1 p24 antigen, affinity analysis by BLI was performed. Antibodies #A, #B and #D were compared with a commercial monoclonal antibody (commercial mAb #1). Anti-mouse Fc specific coated biosensors tips (ForteBio) were used to capture antibodies #A, #B and #D and commercial mAb #1. The gradient of concentrations used for each antibody ranged from 0.1 to 33 nM and each dilution was prepared in phosphate buffer (PBS) containing 0.01% (w/v) bovine serum albumin (BSA) and 0.02% (v/v) of detergent Tween-20. The recorded sensorgrams were fitted using a 1:1 binding model and the equilibrium constant KD was calculated from the ratio of the rate of dissociation and rate of association (kd/ka). The tested antibodies were ranked based on the calculated affinity constants as follows: antibody #B˜antibody #D>antibody #A>commercial mAb #1. Although it was not possible to calculate an accurate KD value for antibodies #B and #D due to the long dissociation curves observed, the data presented shows that the calculated KD values for antibodies #A, #B and #D are lower than the value observed for the commercial mAb #1 (Table 7). This data supports the observation that antibodies #A, #B and #D display higher affinity to HIV-1 p24 than the commercial mAb #1.

TABLE 7
Binding of antibodies #A, #B, #D and commercial
mAb #1to HIV-1 p24 protein by BLI.

| Antibody ID | K_{D} (nM) | Ka (1/Ms) | Kd (1/s) | Rank | Full R² |
|---|---|---|---|---|---|
| A | 0.2 | 2.41 × 10⁵ | 5.90 × 10⁻⁵ | 2 | 0.9982 |
| B | <2.4 × 10⁻⁴ | 4.08 × 10⁵ | <1.0 × 10⁻⁷ | 1 | 0.9993 |
| D | <5.2 × 10⁻⁴ | 1.94 × 10⁵ | <1.0 × 10⁻⁷ | 1 | 0.9986 |
| (commercial | 1.3 | 1.28 × 10⁵ | 1.62 × 10⁻⁴ | 3 | 0.9974 |
| mAb #1) | | | | | |

K_{D}, equilibrium dissociation constant; Ka, association rate constant and Kd, dissociation rate constant.

### Example 6: Binding Capacity of Anti-HIV-1 Antibodies #a, #B and #D

To further evaluate the binding of antibodies #A, #B and #D against HIV-1 p24 antigen, an indirect ELISA assay was completed. Titration curves were generated for each antibody using a starting concentration of 2 μg/mL and performing serial 1:10 dilutions until attaining a lower antibody concentration of 2×10−2 ng/ml. The performance of each antibody was compared to a commercial clone (commercial mAb #2) (FIG. 10, left). The data shows that antibodies #A, #B and #D bind with higher signal-to-noise ratios (S/N) than the commercial antibody, mostly for concentrations between 20 and 2000 ng/ml, and these also display lower EC50 values when compared to the commercial mAb #2 (FIG. 10, right).

### CONCLUSIONS

Functional assays have been performed where anti-HIV-1 antibodies #A, #B and #D have been compared with commercial anti-HIV-1 p24 antibodies from commercial mAbs #1 and #2. The binding affinities and potency of each antibody was evaluated by BLI and indirect ELISA. In both experiments, HIV-1 antibodies #A, #B and #D show better affinity and better EC50 values when compared to the commercial antibodies tested (FIG. 9 and Table 7 for kinetic analysis, and FIG. 10 for ELISA data).

The experimental data presented here demonstrate that the anti-HIV-1 antibodies of the present invention can be used to detect the HIV-1 structural p24 protein. Said antibodies show improved properties in terms of affinity, sensitivity, potency, expression, solubility and manufacturability when compared to similar products on the market and their use in serology can contribute to a reduction in the timeframe between the HIV-1 infection and diagnosis event; therefore, preventing secondary viral transmissions.

Concordance Table

| | | SEQ ID |
|---|---|---|
| Name | Sequence | NO: |
| Light | DIQMTQTTSSLSASLGDRVTINCRASQDISNYLHWYQQKPDG | 1 |
| Chain | TVKLLIYYTSRLHSGVPSRFSGSGSGTDYSLTISNLEKEDIATY | |
| mAb A | FCQQGNSFPWTFGGGTKVEIKRADAAPTVSIFPPSSEQLTSG | |
| | GASVVCFLNNFYPKDINVKWKIDGSERQNGVLNSWTDQDSK | |
| | DSTYSMSSTLTLTKDEYERHNSYTCEATHKTSTSPIVKSFNRN | |
| | EC | |
| Light | DIVLTQSPATLSVTPGDSVSLSCRASQSISDNLHWYRQKSHE | 2 |
| Chain | SPRLLIKYSSQSISGIPSRFSGSGSGTDFTLSINSVETEDFGMY | |
| mAb B | FCQQSNSWPFTFGSGTNLELKRADAAPTVSIFPPSSEQLTSG | |
| | GASVVCFLNNFYPKDINVKWKIDGSERQNGVLNSWTDQDSK | |
| | DSTYSMSSTLTLTKDEYERHNSYTCEATHKTSTSPIVKSFNRN | |
| | EC | |
| Light | DVVMTQTPLSLPVSLGDQASISCRSSQSLVNSDGNTFLQWLL | 3 |
| Chain | QKPGQSPKLLIYKVSNRFSGVPDRFSGSGSGTDFTLRISRVEA | |
| mAb D | EDLGVYFCSQSTHVPWTFGGGTKLEIKRADAAPTVSIFPPSSE | |
| | QLTSGGASVVCFLNNFYPKDINVKWKIDGSERQNGVLNSWTD | |
| | QDSKDSTYSMSSTLTLTKDEYERHNSYTCEATHKTSTSPIVKS | |
| | FNRNEC | |
| Heavy | EVKLVESGGGLVKPGGSLQLSCVASGFTFSSYAMSWVRQTP | 4 |
| Chain | EKGLEWVASITSGGNTYYPDSVKGRFTISRDNAGNILYLQMSS | |
| mAb A | LRSEDTAMFYCAREVLSVPFAYWGQGTLVTVSTAKTTPPSVY | |
| | PLAPGSAAQTNSMVTLGCLVKGYFPEPVTVTWNSGSLSSGV | |
| | HTFPAVLQSDLYTLSSSVTVPSSTWPSETVTCNVAHPASSTK | |
| | VDKKIVPRDCGCKPCICTVPEVSSVFIFPPKPKDVLTITLTPKVT | |
| | CVVVDISKDDPEVQFSWFVDDVEVHTAQTQPREEQFNSTFR | |
| | SVSELPIMHQDWLNGKEFKCRVNSAAFPAPIEKTISKTKGRPK | |
| | APQVYTIPPPKEQMAKDKVSLTCMITDFFPEDITVEWQWNGQ | |
| | PAENYKNTQPIMDTDGSYFVYSKLNVQKSNWEAGNTFTCSVL | |
| | HEGLHNHHTEKSLSHSPG | |
| Heavy | EVOLVESGGGLVKPGGSLKLSCAASGFAFSSYDMSWVRQTP | 5 |
| Chain | DKRLEWVAYITSGVGNLNYLDTVKGRFTISRDNAKNTLYLQM | |
| mAb B | SSLRSEDTAMYFCLRPPSYFGSSYDAMDYWGRGTSVTVSSA | |
| | KTTPPSVYPLAPGSAAQTNSMVTLGCLVKGYFPEPVTVTWNS | |
| | GSLSSGVHTFPAVLQSDLYTLSSSVTVPSSTWPSETVTCNVA | |
| | HPASSTKVDKKIVPRDCGCKPCICTVPEVSSVFIFPPKPKDVLT | |
| | ITLTPKVTCVVVDISKDDPEVQFSWFVDDVEVHTAQTQPREE | |
| | QFNSTFRSVSELPIMHQDWLNGKEFKCRVNSAAFPAPIEKTIS | |
| | KTKGRPKAPQVYTIPPPKEQMAKDKVSLTCMITDFFPEDITVE | |
| | WQWNGQPAENYKNTQPIMDTDGSYFVYSKLNVQKSNWEAG | |
| | NTFTCSVLHEGLHNHHTEKSLSHSPG | |
| Heavy | QIQLQQSGPELVKPGASVKVSCKASGYAFTSYQLYWVKQSH | 6 |
| Chain | GKSLEWIGYIDPYNGGTGYNQKFKGKATLTVDKSSSTAYMHL | |
| mAb D | NSLTSEDSAVYYCASPRWLPAGDYWGQGTSVTVSSAKTTAP | |
| | SVYPLAPVCGDTTGSSVTLGCLVKGYFPEPVTLTWNSGSLSS | |
| | GVHTFPAVLQSDLYTLSSSVTVTSSTWPSQSITCNVAHPASST | |
| | KVDKKIEPRGPTIKPCPPCKCPAPNLLGGPSVFIFPPKIKDVLMI | |
| | SLTPKVTCVVVDVSEDDPDVQISWFVNNVEVHTAQTQTHRED | |
| | YNSTLRVVSALPIQHQDWMSGKEFKCKVNNKDLPAPIERTISK | |
| | PKGSVRAPQVYVLPPPEEEMTKKQVTLTCMVTDFMPEDIYVE | |
| | WTNNGKTELNYKNTEPVLDSDGSYFMYSKLRVEKKNWVERN | |
| | SYSCSVVHEGLHNHHTTKSFSRTPGK | |
| VL Chain | DIQMTQTTSSLSASLGDRVTINCRASQDISNYLHWYQQKPDG | 7 |
| mAb A | TVKLLIYYTSRLHSGVPSRFSGSGSGTDYSLTISNLEKEDIATY | |
| | FCQQGNSFPWTFGGGTKVEIK | |
| VL Chain | DIVLTQSPATLSVTPGDSVSLSCRASQSISDNLHWYROKSHE | 8 |
| mAb B | SPRLLIKYSSQSISGIPSRFSGSGSGTDFTLSINSVETEDFGMY | |
| | FCQQSNSWPFTFGSGTNLELK | |
| VL Chain | DVVMTQTPLSLPVSLGDQASISCRSSQSLVNSDGNTFLQWLL | 9 |
| mAb D | QKPGQSPKLLIYKVSNRFSGVPDRFSGSGSGTDFTLRISRVEA | |
| | EDLGVYFCSQSTHVPWTFGGGTKLEIK | |
| VH Chain | EVKLVESGGGLVKPGGSLQLSCVASGFTFSSYAMSWVRQTP | 10 |
| mAb A | EKGLEWVASITSGGNTYYPDSVKGRFTISRDNAGNILYLQMSS | |
| | LRSEDTAMFYCAREVLSVPFAYWGQGTLVTVST | |
| VH Chain | EVOLVESGGGLVKPGGSLKLSCAASGFAFSSYDMSWVRQTP | 11 |
| mAb B | DKRLEWVAYITSGVGNLNYLDTVKGRFTISRDNAKNTLYLQM | |
| | SSLRSEDTAMYFCLRPPSYFGSSYDAMDYWGRGTSVTVSS | |
| VH Chain | QIQLQQSGPELVKPGASVKVSCKASGYAFTSYQLYWVKQSH | 12 |
| mAb D | GKSLEWIGYIDPYNGGTGYNQKFKGKATLTVDKSSSTAYMHL | |
| | NSLTSEDSAVYYCASPRWLPAGDYWGQGTSVTVSS | |
| CL Chain | RADAAPTVSIFPPSSEQLTSGGASVVCFLNNFYPKDINVKWKI | 13 |
| mAbs A, B | DGSERQNGVLNSWTDQDSKDSTYSMSSTLTLTKDEYERHNS | |
| & D | YTCEATHKTSTSPIVKSFNRNEC | |
| CH Chain | AKTTPPSVYPLAPGSAAQTNSMVTLGCLVKGYFPEPVTVTWN | 14 |
| mAbs A & | SGSLSSGVHTFPAVLQSDLYTLSSSVTVPSSTWPSETVTCNV | |
| B | AHPASSTKVDKKIVPRDCGCKPCICTVPEVSSVFIFPPKPKDVL | |
| | TITLTPKVTCVVVDISKDDPEVQFSWFVDDVEVHTAQTQPREE | |
| | QFNSTFRSVSELPIMHQDWLNGKEFKCRVNSAAFPAPIEKTIS | |
| | KTKGRPKAPQVYTIPPPKEQMAKDKVSLTCMITDFFPEDITVE | |
| | WQWNGQPAENYKNTQPIMDTDGSYFVYSKLNVQKSNWEAG | |
| | NTFTCSVLHEGLHNHHTEKSLSHSPG | |
| CDR 1 VL | RASQDISNYLH | 15 |
| mAb A | | |
| CDR 2 VL | YTSRLHS | 16 |
| mAb A | | |
| CDR 3 VL | QQGNSFPWT | 17 |
| mAb A | | |
| CDR 1 VL | RASQSISDNLH | 18 |
| mAb B | | |
| CDR 2 VL | YSSQSIS | 19 |
| mAb B | | |
| CDR 3 VL | QQSNSWPFT | 20 |
| mAb B | | |
| CDR 1 VL | RSSQSLVNSDGNTFLQ | 21 |
| mAb D | | |
| CDR 2 VL | KVSNRFS | 22 |
| mAb D | | |
| CDR 3 VL | SQSTHVPWT | 23 |
| mAb D | | |
| CDR 1 VH | GFTFSSY | 24 |
| mAb A | | |
| CDR 2 VH | TSGGN | 25 |
| mAb A | | |
| CDR 3 VH | EVLSVPFAY | 26 |
| mAb A | | |
| CDR 1 VH | GFAFSSY | 27 |
| mAb B | | |
| CDR 2 VH | TSGVGN | 28 |
| mAb B | | |
| CDR 3 VH | PPSYFGSSYDAMDY | 29 |
| mAb B | | |
| CDR 1 VH | GYAFTSY | 30 |
| mAb D | | |
| CDR 2 VH | DPYNGG | 31 |
| mAb D | | |
| CDR 3 VH | PRWLPAGDY | 32 |
| mAb D | | |
| Epitope | PIAPGQM | 33 |
| mAb D | | |
| CH Chain | AKTTAPSVYPLAPVCGDTTGSSVTLGCLVKGYFPEPVTLTWN | 34 |
| mAb D | SGSLSSGVHTFPAVLQSDLYTLSSSVTVTSSTWPSQSITCNVA | |
| | HPASSTKVDKKIEPRGPTIKPCPPCKCPAPNLLGGPSVFIFPPK | |
| | IKDVLMISLTPKVTCVVVDVSEDDPDVQISWFVNNVEVHTAQT | |
| | QTHREDYNSTLRVVSALPIQHQDWMSGKEFKCKVNNKDLPA | |
| | PIERTISKPKGSVRAPQVYVLPPPEEEMTKKQVTLTCMVTDFM | |
| | PEDIYVEWTNNGKTELNYKNTEPVLDSDGSYFMYSKLRVEKK | |
| | NWVERNSYSCSVVHEGLHNHHTTKSFSRTPGK | |
| P24 | ALDKIEEEQNKSKKKAQXAAAADAGNSSQVSQNYPIVQNLQG | 35 |
| Antigen | QMVHQAISPRTLNAWVKVVEEKAFSPEVIPMFSALSEGATPQ | |
| Variant 1 | DLNTMLNTVGGHQAAMQMLKETINEEAAEWDRLHPVHAGPIA | |
| | PGQMREPRGSDIAGTTSTLQEQIGWMTNNPPIPVGEIYKRWII | |
| | LGLNKIVRMYSPTSILDIRQGPKEPFRDYVDRFYKTLRAEQAS | |
| | QEVKNWMTETLLVQNANPDCKTILKALGPAATLEEMMTACQG | |
| | VGGPGHKARVLAEAMSQVTNNSATI | |
| P24 | MPIVQNLQGQMVHQAISPRTLNAWVKVVEEKAFSPEVIPMFS | 36 |
| Antigen | ALSEGATPQDLNTMLNTVGGHQAAMQMLKETINEEAAEWRD | |
| Variant 2 | VHPVHAGPIAPGQMREPRGSDIAGTTSTLQEQIGWMTNNPPI | |
| | PVGEIYKRWIILGLNKIVRMYSPTSILDIRQGPKEPFRDYVDRF | |
| | YKTLRAEQASQDVKNWMTETLLVQNANPDCKTILKALGPAAT | |
| | LEEMMTACQGVGGPGHKARVL | |
| Nucleotide | ATGGAGACCGACACCCTGCTGCTCTGGGTGCTGCTGCTCT | 37 |
| sequence | GGGTGCCCGGCTCCACCGGAGACATCCAGATGACCCAGA | |
| for VL | CCACCAGCTCCCTGAGCGCCAGCCTGGGCGACAGGGTGA | |
| mAb A | CCATCAACTGCAGGGCCAGCCAGGACATCAGCAACTACCT | |
| | GCACTGGTATCAACAGAAGCCCGACGGCACGGTGAAACTG | |
| | CTGATCTACTATACCAGCAGGCTGCACAGCGGCGTGCCCA | |
| | GCCGCTTCTCCGGTAGCGGCAGCGGCACCGACTACTCTCT | |
| | GACCATTAGCAACCTGGAGAAGGAGGACATTGCCACCTAC | |
| | TTCTGTCAGCAGGGCAACAGCTTCCCCTGGACCTTCGGCG | |
| | GAGGAACCAAAGTGGAAATCAAGCGGGCAGATGCTGCACC | |
| | AACTGTATCCATCTTCCCACCATCCAGTGAGCAGTTAACAT | |
| | CTGGAGGTGCCTCAGTCGTGTGCTTCTTGAACAACTTCTAC | |
| | CCCAAAGACATCAATGTCAAGTGGAAGATTGATGGCAGTG | |
| | AACGACAAAATGGCGTCCTGAACAGTTGGACTGATCAGGA | |
| | CAGCAAAGACAGCACCTACAGCATGAGCAGCACCCTCACG | |
| | TTGACCAAGGACGAGTATGAACGACATAACAGCTATACCTG | |
| | TGAGGCCACTCACAAGACATCAACTTCACCCATTGTCAAGA | |
| | GCTTCAACAGGAATGAGTGTTGA | |
| Nucleotide | ATGGACCCCAAGGGCAGCCTGAGCTGGAGAATCCTGCTGT | 38 |
| sequence | TCCTGAGCCTGGCCTTCGAGCTGAGCTACGGCGAGGTGAA | |
| for VH | GCTCGTGGAGAGCGGCGGTGGCCTGGTTAAGCCTGGGGG | |
| mAb A | AAGCCTGCAGCTGAGCTGCGTGGCCAGCGGCTTCACGTTC | |
| | AGCAGCTACGCCATGAGCTGGGTGAGGCAGACCCCCGAG | |
| | AAGGGCCTGGAGTGGGTGGCAAGCATCACCAGCGGGGGT | |
| | AACACCTACTACCCCGACAGCGTGAAGGGCAGGTTCACCA | |
| | TCAGCAGGGACAACGCTGGCAACATCCTGTACCTGCAGAT | |
| | GAGCAGCCTGAGGAGCGAGGACACCGCCATGTTCTACTGC | |
| | GCCAGGGAGGTGCTGAGCGTCCCCTTCGCCTACTGGGGC | |
| | CAGGGCACCCTGGTCACAGTGAGCACCGCCAAGACCACTC | |
| | CACCTTCCGTGTACCCTCTGGCTCCTGGATCTGCCGCCCA | |
| | GACCAACTCCATGGTCACCCTGGGCTGCCTCGTGAAGGGC | |
| | TACTTCCCTGAGCCTGTGACCGTGACCTGGAACTCCGGCT | |
| | CTCTGTCCTCTGGCGTGCACACCTTCCCTGCCGTGCTGCA | |
| | GTCCGACCTGTACACCCTGTCCTCCAGCGTGACCGTGCCT | |
| | TCCTCTACCTGGCCCTCCGAGACAGTGACCTGCAACGTGG | |
| | CCCACCCTGCCAGCTCTACCAAGGTGGACAAGAAAATCGT | |
| | GCCCCGGGACTGCGGCTGCAAGCCCTGTATCTGTACCGTG | |
| | CCCGAGGTGTCCTCCGTGTTCATCTTCCCACCCAAGCCCA | |
| | AGGACGTGCTGACCATCACCCTGACCCCCAAAGTGACCTG | |
| | TGTGGTGGTGGACATCTCCAAGGACGACCCCGAGGTGCA | |
| | GTTCAGTTGGTTCGTGGACGACGTGGAAGTGCACACCGCT | |
| | CAGACCCAGCCCAGAGAGGAACAGTTCAACTCCACCTTCA | |
| | GATCCGTGTCCGAGCTGCCCATCATGCACCAGGACTGGCT | |
| | GAACGGCAAAGAGTTCAAGTGCAGAGTGAACTCCGCCGCC | |
| | TTCCCAGCCCCCATCGAAAAGACCATCAGCAAGACCAAGG | |
| | GCAGACCCAAGGCCCCCCAGGTGTACACAATCCCGCCACC | |
| | CAAAGAACAGATGGCCAAGGACAAGGTGTCCCTGACCTGC | |
| | ATGATCACCGATTTCTTCCCAGAGGATATTACCGTGGAATG | |
| | GCAGTGGAACGGCCAGCCCGCCGAGAACTACAAGAACAC | |
| | CCAGCCTATCATGGACACCGACGGCTCCTACTTCGTGTAC | |
| | TCCAAGCTGAACGTGCAGAAGTCCAACTGGGAGGCCGGCA | |
| | ACACCTTCACCTGTAGCGTGCTGCACGAGGGCCTGCACAA | |
| | TCACCACACCGAGAAGTCCCTGTCCCACTCCCCTGGCTAG | |
| Nucleotide | ATGGAGACCGACACCCTGCTGCTCTGGGTGCTGCTGCTCT | 39 |
| sequence | GGGTGCCCGGCTCCACCGGAGACATCGTGCTGACCCAGA | |
| for VL | GCCCTGCCACCCTGAGCGTGACCCCTGGCGACAGCGTGA | |
| mAb B | GCCTGAGCTGCAGGGCCAGCCAGAGCATTAGCGACAACCT | |
| | GCACTGGTACAGGCAGAAAAGCCACGAAAGCCCCAGGCTT | |
| | CTGATCAAGTACAGCAGCCAAAGCATCTCAGGCATCCCCA | |
| | GCAGGTTCAGTGGGAGCGGCAGCGGCACCGACTTCACCC | |
| | TGTCCATCAACAGCGTTGAGACCGAGGACTTCGGCATGTA | |
| | CTTCTGCCAGCAGAGCAACAGCTGGCCGTTTACCTTCGGC | |
| | TCCGGCACTAACCTGGAGCTGAAGCGGGCAGATGCTGCAC | |
| | CAACTGTATCCATCTTCCCACCATCCAGTGAGCAGTTAACA | |
| | TCTGGAGGTGCCTCAGTCGTGTGCTTCTTGAACAACTTCTA | |
| | CCCCAAAGACATCAATGTCAAGTGGAAGATTGATGGCAGT | |
| | GAACGACAAAATGGCGTCCTGAACAGTTGGACTGATCAGG | |
| | ACAGCAAAGACAGCACCTACAGCATGAGCAGCACCCTCAC | |
| | GTTGACCAAGGACGAGTATGAACGACATAACAGCTATACCT | |
| | GTGAGGCCACTCACAAGACATCAACTTCACCCATTGTCAAG | |
| | AGCTTCAACAGGAATGAGTGTTGA | |
| Nucleotide | ATGGACCCCAAGGGCAGCCTGAGCTGGAGAATCCTGCTGT | 40 |
| sequence | TCCTGAGCCTGGCCTTCGAGCTGAGCTACGGCGAGGTGCA | |
| for VH | GCTGGTGGAGAGCGGGGGTGGACTTGTGAAGCCCGGTGG | |
| mAb B | CTCACTGAAGCTGAGCTGCGCGGCAAGCGGCTTCGCCTTC | |
| | AGCAGCTACGACATGAGCTGGGTGAGGCAGACCCCCGAC | |
| | AAGAGGCTGGAGTGGGTGGCCTACATCACCAGTGGCGTG | |
| | GGCAACCTGAACTACCTGGACACCGTGAAGGGCAGGTTCA | |
| | CCATCAGCAGGGACAACGCCAAGAACACCCTGTACCTGCA | |
| | GATGAGCAGCTTGAGGAGCGAAGACACCGCCATGTACTTC | |
| | TGCCTGAGACCGCCCAGCTACTTCGGCTCTAGCTATGATG | |
| | CCATGGACTACTGGGGCAGGGGTACTAGCGTGACCGTGA | |
| | GCTCTGCCAAGACCACTCCACCTTCCGTGTACCCTCTGGC | |
| | TCCTGGATCTGCCGCCCAGACCAACTCCATGGTCACCCTG | |
| | GGCTGCCTCGTGAAGGGCTACTTCCCTGAGCCTGTGACCG | |
| | TGACCTGGAACTCCGGCTCTCTGTCCTCTGGCGTGCACAC | |
| | CTTCCCTGCCGTGCTGCAGTCCGACCTGTACACCCTGTCC | |
| | TCCAGCGTGACCGTGCCTTCCTCTACCTGGCCCTCCGAGA | |
| | CAGTGACCTGCAACGTGGCCCACCCTGCCAGCTCTACCAA | |
| | GGTGGACAAGAAAATCGTGCCCCGGGACTGCGGCTGCAA | |
| | GCCCTGTATCTGTACCGTGCCCGAGGTGTCCTCCGTGTTC | |
| | ATCTTCCCACCCAAGCCCAAGGACGTGCTGACCATCACCC | |
| | TGACCCCCAAAGTGACCTGTGTGGTGGTGGACATCTCCAA | |
| | GGACGACCCCGAGGTGCAGTTCAGTTGGTTCGTGGACGAC | |
| | GTGGAAGTGCACACCGCTCAGACCCAGCCCAGAGAGGAA | |
| | CAGTTCAACTCCACCTTCAGATCCGTGTCCGAGCTGCCCAT | |
| | CATGCACCAGGACTGGCTGAACGGCAAAGAGTTCAAGTGC | |
| | AGAGTGAACTCCGCCGCCTTCCCAGCCCCCATCGAAAAGA | |
| | CCATCAGCAAGACCAAGGGCAGACCCAAGGCCCCCCAGG | |
| | TGTACACAATCCCGCCACCCAAAGAACAGATGGCCAAGGA | |
| | CAAGGTGTCCCTGACCTGCATGATCACCGATTTCTTCCCAG | |
| | AGGATATTACCGTGGAATGGCAGTGGAACGGCCAGCCCGC | |
| | CGAGAACTACAAGAACACCCAGCCTATCATGGACACCGAC | |
| | GGCTCCTACTTCGTGTACTCCAAGCTGAACGTGCAGAAGT | |
| | CCAACTGGGAGGCCGGCAACACCTTCACCTGTAGCGTGCT | |
| | GCACGAGGGCCTGCACAATCACCACACCGAGAAGTCCCTG | |
| | TCCCACTCCCCTGGCTAG | |
| Nucleotide | ATGGAGACCGACACCCTGCTGCTCTGGGTGCTGCTGCTCT | 41 |
| sequence | GGGTGCCCGGCTCCACCGGAGACGTGGTGATGACCCAGA | |
| for VL | CACCCCTGAGTCTGCCCGTGAGCTTGGGCGACCAGGCCA | |
| mAb D | GCATCAGCTGTAGGAGCTCACAGAGCCTGGTGAACAGCGA | |
| | CGGCAACACCTTCCTGCAGTGGCTCCTGCAAAAACCCGGC | |
| | CAAAGCCCGAAGCTGCTTATATACAAGGTGAGCAATAGGTT | |
| | CAGTGGTGTGCCCGACCGCTTCAGCGGCAGCGGTAGCGG | |
| | CACCGACTTCACCCTGAGGATCAGCAGGGTGGAGGCCGA | |
| | GGACCTGGGCGTGTACTTCTGCAGCCAGAGCACCCACGTG | |
| | CCCTGGACCTTCGGCGGAGGAACCAAACTGGAAATCAAGC | |
| | GGGCAGATGCTGCACCAACTGTATCCATCTTCCCACCATC | |
| | CAGTGAGCAGTTAACATCTGGAGGTGCCTCAGTCGTGTGC | |
| | TTCTTGAACAACTTCTACCCCAAAGACATCAATGTCAAGTG | |
| | GAAGATTGATGGCAGTGAACGACAAAATGGCGTCCTGAAC | |
| | AGTTGGACTGATCAGGACAGCAAAGACAGCACCTACAGCA | |
| | TGAGCAGCACCCTCACGTTGACCAAGGACGAGTATGAACG | |
| | ACATAACAGCTATACCTGTGAGGCCACTCACAAGACATCAA | |
| | CTTCACCCATTGTCAAGAGCTTCAACAGGAATGAGTGTTGA | |
| Nucleotide | ATGGAGACCGACACCCTGCTGCTCTGGGTGCTGCTGCTCT | 42 |
| sequence | GGGTGCCCGGCTCCACCGGACAGATCCAGCTGCAACAAA | |
| for VH | GCGGCCCTGAGCTGGTGAAGCCCGGTGCTAGCGTGAAGG | |
| mAb D | TGAGCTGTAAGGCAAGCGGCTACGCCTTCACAAGTTACCA | |
| | GCTGTACTGGGTAAAGCAAAGCCACGGCAAGAGCCTGGAG | |
| | TGGATCGGCTATATCGACCCCTACAACGGCGGCACCGGCT | |
| | ACAACCAGAAGTTCAAGGGTAAGGCCACCTTGACCGTGGA | |
| | CAAGAGCAGCAGCACCGCCTACATGCATCTGAACAGCCTG | |
| | ACCAGCGAGGACAGCGCCGTGTACTACTGCGCCAGCCCC | |
| | AGGTGGCTTCCCGCTGGCGACTACTGGGGCCAGGGCACC | |
| | AGCGTGACTGTGAGCTCTGCTAAAACAACAGCCCCATCGG | |
| | TCTATCCACTGGCCCCTGTGTGTGGAGATACAACTGGCTC | |
| | CTCGGTGACTCTAGGATGCCTGGTCAAGGGTTATTTCCCT | |
| | GAGCCAGTGACCTTGACCTGGAACTCTGGTTCCCTGTCCA | |
| | GTGGTGTGCACACCTTCCCAGCTGTCCTGCAGTCTGACCT | |
| | CTACACCCTCAGCTCAAGCGTGACTGTAACCAGCTCGACC | |
| | TGGCCCAGCCAGTCCATCACCTGCAATGTGGCCCACCCGG | |
| | CAAGCAGCACCAAGGTGGACAAGAAAATTGAGCCCAGAGG | |
| | GCCCACAATCAAGCCCTGTCCTCCATGCAAATGCCCAGCA | |
| | CCTAACCTCTTGGGTGGACCATCCGTCTTCATCTTCCCTCC | |
| | AAAGATCAAGGATGTACTCATGATCTCCCTGAGCCCCATAG | |
| | TCACATGTGTAGTCGTTGATGTGAGCGAGGATGACCCAGA | |
| | TGTCCAGATCAGCTGGTTTGTGAACAACGTGGAAGTGCAC | |
| | ACTGCTCAGACACAGACGCATAGAGAGGATTACAACAGTA | |
| | CTCTCCGGGTTGTCAGTGCCCTCCCCATCCAGCACCAGGA | |
| | CTGGATGAGTGGCAAGGAGTTCAAATGCAAGGTCAACAAC | |
| | AAAGACCTCCCAGCGCCCATCGAGAGAACCATCTCAAAAC | |
| | CCAAAGGGTCAGTAAGAGCTCCACAGGTATATGTCTTGCCT | |
| | CCACCAGAAGAGGAGATGACTAAGAAACAGGTCACTCTGA | |
| | CCTGCATGGTCACAGACTTCATGCCTGAAGACATTTACGTG | |
| | GAGTGGACCAACAACGGGAAAACAGAGCTAAACTACAAGA | |
| | ACACTGAACCAGTCCTGGACTCTGATGGTTCTTACTTCATG | |
| | TACAGCAAGCTGAGAGTGGAGAAGAAGAACTGGGTGGAGA | |
| | GAAATAGCTACTCCTGTTCAGTGGTCCACGAGGGTCTGCA | |
| | CAATCACCACACGACTAAGAGCTTCTCCCGGACTCCGGGT | |
| | TAG | |

*Unless otherwise stated all CDR sequences have been derived according to Chothia utilizing the Abysis database.

1. An anti-HIV-1 antibody or fragment thereof comprising a light chain comprising complementary determining regions L-CDR1, L-CDR2 and L-CDR3, wherein
the amino acid sequence of L-CDR1 is selected from the group consisting of SEQ ID NO: 15, SEQ ID NO: 18, SEQ ID NO: 21, and a sequence that differs from anyone of SEQ ID NOs: 15, 18, or 21 by one or two substitutions, deletions, or additions,
the amino acid sequence of L-CDR2 is selected from the group consisting of SEQ ID NO: 16, SEQ ID NO: 19, SEQ ID NO: 22, and a sequence that differs from anyone of SEQ ID NOs: 16, 19, or 22 by one or two substitutions, deletions, or additions, and
the amino acid sequence of L-CDR3 is selected from the group consisting of SEQ ID NO: 17, SEQ ID NO: 20, SEQ ID NO: 23, and a sequence that differs from anyone of SEQ ID NOs: 17, 20, or 23 by one or two substitutions, deletions, or additions.

2. The anti-HIV-1 antibody according to claim 1, wherein said antibody comprises a heavy chain comprising complementary determining regions H-CDR1, H-CDR2 and H-CDR3, wherein
the amino acid sequence of H-CDR1 is selected from the group consisting of SEQ ID NO: 24, SEQ ID NO: 27, SEQ ID NO: 30, and a sequence that differs from anyone of SEQ ID NOs: 24, 27, or 30 by one or two substitutions, deletions, or additions,
the amino acid sequence of H-CDR2 is selected from the group consisting of SEQ ID NO: 25, SEQ ID NO: 28, SEQ ID NO: 31, and a sequence that differs from anyone of SEQ ID NOs: 25, 28, or 31 by one or two substitutions, deletions, or additions, and
the amino acid sequence of H-CDR3 is selected from the group consisting of SEQ ID NO: 26, SEQ ID NO: 29, SEQ ID NO: 32, and a sequence that differs from anyone of SEQ ID NOs: 26, 29, or 32 by one or two substitutions, deletions, or additions.

3. The anti-HIV-1 antibody according to claim 1, wherein the variable region of the light chain comprises a sequence having about 90% homology with the amino acid sequence of SEQ ID NO: 7, SEQ ID NO: 8, or SEQ ID NO: 9.

4. (canceled)

5. The anti-HIV-1 antibody according to claim 1, wherein the variable region of the heavy chain comprises a sequence having about 90% homology with the amino acid sequence of SEQ ID NO: 10, SEQ ID NO: 11, or SEQ ID NO: 12.

6. (canceled)

7. The anti-HIV-1 antibody according to claim 1, wherein the amino acid sequence of L-CDR1 comprises SEQ ID NO: or a sequence that differs from SEQ ID NO: 15 by one or two substitutions, deletions, or additions,
the amino acid sequence of L-CDR2 comprises SEQ ID NO: 16 or a sequence that differs from SEQ ID NO: 16 by one or two substitutions, deletions, or additions, and
the amino acid sequence of L-CDR3 comprises SEQ ID NO: 17 or a sequence that differs from SEQ ID NO: 17 by one or two substitutions, deletions, or additions.

8. The anti-HIV-1 antibody according to claim 1, wherein the amino acid sequence of L-CDR1 comprises SEQ ID NO: 18 or a sequence that differs from SEQ ID NO: 18 by one or two substitutions, deletions, or additions,
the amino acid sequence of L-CDR2 comprises SEQ ID NO: 19 or a sequence that differs from SEQ ID NO: 19 by one or two substitutions, deletions, or additions, and
the amino acid sequence of L-CDR3 comprises SEQ ID NO: 20 or a sequence that differs from SEQ ID NO: 20 by one or two substitutions, deletions, or additions.

9. The anti-HIV-1 antibody according to claim 1, wherein the amino acid sequence of L-CDR1 comprises SEQ ID NO: 21 or a sequence that differs from SEQ ID NO: 21 by one or two substitutions, deletions, or additions,
the amino acid sequence of L-CDR2 comprises SEQ ID NO: 22 or a sequence that differs from SEQ ID NO: 22 by one or two substitutions, deletions, or additions, and
the amino acid sequence of L-CDR3 comprises SEQ ID NO: 23. or a sequence that differs from SEQ ID NO: 23 by one or two substitutions, deletions, or additions.

10-15. (canceled)

16. The anti-HIV-1 antibody according to claim 1, wherein the light chain of said antibody comprises the amino acid sequence selected from the group consisting of SEQ ID NO: 1, SEQ ID NO: 2, or SEQ ID NO: 3.

17. The anti-HIV-1 antibody according to claim 1, wherein the heavy chain of said antibody comprises the amino acid sequence selected from the group consisting of SEQ ID NO: 4, SEQ ID NO: 5, or SEQ ID NO: 6.

18. (canceled)

19. The anti-HIV-1 antibody according to claim 1, wherein the antibody is a monoclonal antibody or a recombinant antibody, or a fragment thereof.

20. (canceled)

21. The anti-HIV-1 antibody according to claim 1, wherein the antibody fragment is selected from variable fragments (Fv), single-chain Fvs (scFv), bispecific antibodies (sc(Fv)2), single chain antibodies, single domain antibodies, Fab fragments, F(ab′)2 fragments, Fab′ fragments, disulfide-linked Fv (dsFv), chemically conjugated Fv (ccFv), diabodies, anti-idiotypic (anti-Id) antibodies, affibodies, nanobodies, and unibodies.

22. The anti-HIV-1 antibody according to claim 1, wherein the antibody comprises a constant region of the murine IgG1 class or the murine IgG2a class.

23. The anti-HIV-1 antibody according to claim 1, wherein the antibody is bound to a solid support.

24. (canceled)

25. A nucleic acid comprising a nucleotide sequence encoding the anti-HIV-1 antibody according to claim 1, a promoter operably linked to the nucleotide sequence and a selectable marker.

26. (canceled)

27. A composition comprising the anti-HIV-1 antibody of claim 1, and a solid support, wherein the anti-HIV-1 antibody is covalently or non-covalently bound to the solid support.

28. The composition of claim 27, wherein the solid support comprises a particle, a bead, a membrane, a surface, a polypeptide chip, a microtiter plate, or the solid-phase of a chromatography column.

29. A kit for detecting the presence of HIV-1 in a sample, the kit comprising at least one anti-HIV-1 antibody according to claim 1 and a solid support, wherein said at least one antibody is covalently or non-covalently bound to a solid support.

30. An anti-HIV-1 antibody is characterized that it specifically binds to an epitope of HIV-1 p24 protein comprising the amino acid sequence of SEQ ID NO: 33.

31. The anti-HIV-1 antibody according to claim 30;
wherein the amino acid sequence of L-CDR1 comprises SEQ ID NO: 21 or a sequence that differs from SEQ ID NO: 21 by one or two substitutions, deletions, or additions;
wherein the amino acid sequence of L-CDR2 comprises SEQ ID NO: 22 or a sequence that differs from SEQ ID NO: 22 by one or two substitutions, deletions, or additions; and
wherein the amino acid sequence of L-CDR3 comprises SEQ ID NO: 23. or a sequence that differs from SEQ ID NO: 23 by one or two substitutions, deletions, or additions.

32. The anti-HIV-1 antibody according to claim 30,
wherein the amino acid sequence of H-CDR1 comprises SEQ ID NO: 30 or a sequence that differs from SEQ ID NO: 30 by one or two substitutions, deletions, or additions;
wherein the amino acid sequence of H-CDR2 comprises SEQ ID NO: 31 or a sequence that differs from SEQ ID NO: 31 by one or two substitutions, deletions, or additions; and
wherein the amino acid sequence of H-CDR3 comprises SEQ ID NO: 32 or a sequence that differs from SEQ ID NO: 32 by one or two substitutions, deletions, or additions.


SEQUENCE LISTING (42 sequences)

SEQ ID NO: 1 - protein, 214 aa, Artificial Sequence
  Light chain mAb A
DIQMTQTTSSLSASLGDRVTINCRASQDISNYLHWYQQKPDGTVKLLIYYTSRLHSGVPSRFSGSGSGTDYSLTISNLEKEDIATYFCQQGNSFPWTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 2 - protein, 214 aa, Artificial Sequence
  Light chain mAb B
DIVLTQSPATLSVTPGDSVSLSCRASQSISDNLHWYRQKSHESPRLLIKYSSQSISGIPSRFSGSGSGTDFTLSINSVETEDFGMYFCQQSNSWPFTFGSGTNLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 219 aa, Artificial Sequence
  Light chain mAb D
DVVMTQTPLSLPVSLGDQASISCRSSQSLVNSDGNTFLQWLLQKPGQSPKLLIYKVSNRFSGVPDRFSGSGSGTDFTLRISRVEAEDLGVYFCSQSTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 4 - protein, 440 aa, Artificial Sequence
  Heavy chain mAb A
EVKLVESGGGLVKPGGSLQLSCVASGFTFSSYAMSWVRQTPEKGLEWVASITSGGNTYYPDSVKGRFTISRDNAGNILYLQMSSLRSEDTAMFYCAREVLSVPFAYWGQGTLVTVSTAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 5 - protein, 446 aa, Artificial Sequence
  Heavy chain mAb B
EVQLVESGGGLVKPGGSLKLSCAASGFAFSSYDMSWVRQTPDKRLEWVAYITSGVGNLNYLDTVKGRFTISRDNAKNTLYLQMSSLRSEDTAMYFCLRPPSYFGSSYDAMDYWGRGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 6 - protein, 448 aa, Artificial Sequence
  Heavy chain mAb D
QIQLQQSGPELVKPGASVKVSCKASGYAFTSYQLYWVKQSHGKSLEWIGYIDPYNGGTGYNQKFKGKATLTVDKSSSTAYMHLNSLTSEDSAVYYCASPRWLPAGDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLTPKVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 7 - protein, 107 aa, Artificial Sequence
  VL chain mAb A
DIQMTQTTSSLSASLGDRVTINCRASQDISNYLHWYQQKPDGTVKLLIYYTSRLHSGVPSRFSGSGSGTDYSLTISNLEKEDIATYFCQQGNSFPWTFGGGTKVEIK

SEQ ID NO: 8 - protein, 107 aa, Artificial Sequence
  VL chain mAb B
DIVLTQSPATLSVTPGDSVSLSCRASQSISDNLHWYRQKSHESPRLLIKYSSQSISGIPSRFSGSGSGTDFTLSINSVETEDFGMYFCQQSNSWPFTFGSGTNLELK

SEQ ID NO: 9 - protein, 112 aa, Artificial Sequence
  VL chain mAb D
DVVMTQTPLSLPVSLGDQASISCRSSQSLVNSDGNTFLQWLLQKPGQSPKLLIYKVSNRFSGVPDRFSGSGSGTDFTLRISRVEAEDLGVYFCSQSTHVPWTFGGGTKLEIK

SEQ ID NO: 10 - protein, 117 aa, Artificial Sequence
  VH chain mAb A
EVKLVESGGGLVKPGGSLQLSCVASGFTFSSYAMSWVRQTPEKGLEWVASITSGGNTYYPDSVKGRFTISRDNAGNILYLQMSSLRSEDTAMFYCAREVLSVPFAYWGQGTLVTVST

SEQ ID NO: 11 - protein, 123 aa, Artificial Sequence
  VH chain mAb B
EVQLVESGGGLVKPGGSLKLSCAASGFAFSSYDMSWVRQTPDKRLEWVAYITSGVGNLNYLDTVKGRFTISRDNAKNTLYLQMSSLRSEDTAMYFCLRPPSYFGSSYDAMDYWGRGTSVTVSS

SEQ ID NO: 12 - protein, 118 aa, Artificial Sequence
  VH chain mAb D
QIQLQQSGPELVKPGASVKVSCKASGYAFTSYQLYWVKQSHGKSLEWIGYIDPYNGGTGYNQKFKGKATLTVDKSSSTAYMHLNSLTSEDSAVYYCASPRWLPAGDYWGQGTSVTVSS

SEQ ID NO: 13 - protein, 107 aa, Artificial Sequence
  CL chain mAbs A,B and D
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 323 aa, Artificial Sequence
  CH Chain mAbs A and B
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 15 - protein, 11 aa, Artificial Sequence
  CDR 1 VL mAb A
RASQDISNYLH

SEQ ID NO: 16 - protein, 7 aa, Artificial Sequence
  CDR 2 VL mAb A
YTSRLHS

SEQ ID NO: 17 - protein, 9 aa, Artificial Sequence
  CDR 3 VL mAb A
QQGNSFPWT

SEQ ID NO: 18 - protein, 11 aa, Artificial Sequence
  CDR 1 VL mAb B
RASQSISDNLH

SEQ ID NO: 19 - protein, 7 aa, Artificial Sequence
  CDR 2 VL mAb B
YSSQSIS

SEQ ID NO: 20 - protein, 9 aa, CDR 3 VL mAb B
  Tissue plasminogen activator signal peptide
QQSNSWPFT

SEQ ID NO: 21 - protein, 16 aa, Artificial Sequence
  CDR 1 VL mAb D
RSSQSLVNSDGNTFLQ

SEQ ID NO: 22 - protein, 7 aa, Artificial Sequence
  CDR 2 VL mAb D
KVSNRFS

SEQ ID NO: 23 - protein, 9 aa, Artificial Sequence
  CDR 3 VL mAb D
SQSTHVPWT

SEQ ID NO: 24 - protein, 7 aa, Artificial Sequence
  CDR 1 VH mAb A
GFTFSSY

SEQ ID NO: 25 - protein, 5 aa, Artificial Sequence
  CDR 2 VH mAb A
TSGGN

SEQ ID NO: 26 - protein, 9 aa, Artificial Sequence
  CDR 3 VH mAb A
EVLSVPFAY

SEQ ID NO: 27 - protein, 7 aa, Artificial Sequence
  CDR 1 VH mAb B
GFAFSSY

SEQ ID NO: 28 - protein, 6 aa, Artificial Sequence
  CDR 2 VH mAb B
TSGVGN

SEQ ID NO: 29 - protein, 14 aa, Artificial Sequence
  CDR 3 VH mAb B
PPSYFGSSYDAMDY

SEQ ID NO: 30 - protein, 7 aa, Artificial Sequence
  CDR 1 VH mAb D
GYAFTSY

SEQ ID NO: 31 - protein, 6 aa, Artificial Sequence
  CDR 2 VH mAb D
DPYNGG

SEQ ID NO: 32 - protein, 9 aa, Artificial Sequence
  CDR 3 VH mAb D
PRWLPAGDY

SEQ ID NO: 33 - protein, 7 aa, Artificial Sequence
  Epitope mAb D
PIAPGQM

SEQ ID NO: 34 - protein, 330 aa, Artificial Sequence
  CH Chain mAb D
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLTPKVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 35 - protein, 279 aa, Artificial Sequence
  P24 Antigen Variant 1
  SITE (18)..(18): Xaa is any amino acid
ALDKIEEEQNKSKKKAQXAAAADAGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNNSATI

SEQ ID NO: 36 - protein, 232 aa, Artificial Sequence
  P24 Antigen Variant 2
MPIVQNLQGQMVHQAISPRTLNAWVKVVEEKAFSPEVIPMFSALSEGATPQDLNTMLNTVGGHQAAMQMLKETINEEAAEWRDVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQDVKNWMTETLLVQNANPDCKTILKALGPAATLEEMMTACQGVGGPGHKARVL

SEQ ID NO: 37 - DNA, 705 bp, Artificial Sequence
  Nucleotide sequence for VL mAb A
atggagaccgacaccctgctgctctgggtgctgctgctctgggtgcccggctccaccggagacatccagatgacccagaccaccagctccctgagcgccagcctgggcgacagggtgaccatcaactgcagggccagccaggacatcagcaactacctgcactggtatcaacagaagcccgacggcacggtgaaactgctgatctactataccagcaggctgcacagcggcgtgcccagccgcttctccggtagcggcagcggcaccgactactctctgaccattagcaacctggagaaggaggacattgccacctacttctgtcagcagggcaacagcttcccctggaccttcggcggaggaaccaaagtggaaatcaagcgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 38 - DNA, 1395 bp, Artificial Sequence
  Nucleotide sequence for VH mAb A
atggaccccaagggcagcctgagctggagaatcctgctgttcctgagcctggccttcgagctgagctacggcgaggtgaagctcgtggagagcggcggtggcctggttaagcctgggggaagcctgcagctgagctgcgtggccagcggcttcacgttcagcagctacgccatgagctgggtgaggcagacccccgagaagggcctggagtgggtggcaagcatcaccagcgggggtaacacctactaccccgacagcgtgaagggcaggttcaccatcagcagggacaacgctggcaacatcctgtacctgcagatgagcagcctgaggagcgaggacaccgccatgttctactgcgccagggaggtgctgagcgtccccttcgcctactggggccagggcaccctggtcacagtgagcaccgccaagaccactccaccttccgtgtaccctctggctcctggatctgccgcccagaccaactccatggtcaccctgggctgcctcgtgaagggctacttccctgagcctgtgaccgtgacctggaactccggctctctgtcctctggcgtgcacaccttccctgccgtgctgcagtccgacctgtacaccctgtcctccagcgtgaccgtgccttcctctacctggccctccgagacagtgacctgcaacgtggcccaccctgccagctctaccaaggtggacaagaaaatcgtgccccgggactgcggctgcaagccctgtatctgtaccgtgcccgaggtgtcctccgtgttcatcttcccacccaagcccaaggacgtgctgaccatcaccctgacccccaaagtgacctgtgtggtggtggacatctccaaggacgaccccgaggtgcagttcagttggttcgtggacgacgtggaagtgcacaccgctcagacccagcccagagaggaacagttcaactccaccttcagatccgtgtccgagctgcccatcatgcaccaggactggctgaacggcaaagagttcaagtgcagagtgaactccgccgccttcccagcccccatcgaaaagaccatcagcaagaccaagggcagacccaaggccccccaggtgtacacaatcccgccacccaaagaacagatggccaaggacaaggtgtccctgacctgcatgatcaccgatttcttcccagaggatattaccgtggaatggcagtggaacggccagcccgccgagaactacaagaacacccagcctatcatggacaccgacggctcctacttcgtgtactccaagctgaacgtgcagaagtccaactgggaggccggcaacaccttcacctgtagcgtgctgcacgagggcctgcacaatcaccacaccgagaagtccctgtcccactcccctggctag

SEQ ID NO: 39 - DNA, 705 bp, Artificial Sequence
  Nucleotide sequence for VL mAb B
atggagaccgacaccctgctgctctgggtgctgctgctctgggtgcccggctccaccggagacatcgtgctgacccagagccctgccaccctgagcgtgacccctggcgacagcgtgagcctgagctgcagggccagccagagcattagcgacaacctgcactggtacaggcagaaaagccacgaaagccccaggcttctgatcaagtacagcagccaaagcatctcaggcatccccagcaggttcagtgggagcggcagcggcaccgacttcaccctgtccatcaacagcgttgagaccgaggacttcggcatgtacttctgccagcagagcaacagctggccgtttaccttcggctccggcactaacctggagctgaagcgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 40 - DNA, 1413 bp, Artificial Sequence
  Nucleotide sequence for VH mAb B
atggaccccaagggcagcctgagctggagaatcctgctgttcctgagcctggccttcgagctgagctacggcgaggtgcagctggtggagagcgggggtggacttgtgaagcccggtggctcactgaagctgagctgcgcggcaagcggcttcgccttcagcagctacgacatgagctgggtgaggcagacccccgacaagaggctggagtgggtggcctacatcaccagtggcgtgggcaacctgaactacctggacaccgtgaagggcaggttcaccatcagcagggacaacgccaagaacaccctgtacctgcagatgagcagcttgaggagcgaagacaccgccatgtacttctgcctgagaccgcccagctacttcggctctagctatgatgccatggactactggggcaggggtactagcgtgaccgtgagctctgccaagaccactccaccttccgtgtaccctctggctcctggatctgccgcccagaccaactccatggtcaccctgggctgcctcgtgaagggctacttccctgagcctgtgaccgtgacctggaactccggctctctgtcctctggcgtgcacaccttccctgccgtgctgcagtccgacctgtacaccctgtcctccagcgtgaccgtgccttcctctacctggccctccgagacagtgacctgcaacgtggcccaccctgccagctctaccaaggtggacaagaaaatcgtgccccgggactgcggctgcaagccctgtatctgtaccgtgcccgaggtgtcctccgtgttcatcttcccacccaagcccaaggacgtgctgaccatcaccctgacccccaaagtgacctgtgtggtggtggacatctccaaggacgaccccgaggtgcagttcagttggttcgtggacgacgtggaagtgcacaccgctcagacccagcccagagaggaacagttcaactccaccttcagatccgtgtccgagctgcccatcatgcaccaggactggctgaacggcaaagagttcaagtgcagagtgaactccgccgccttcccagcccccatcgaaaagaccatcagcaagaccaagggcagacccaaggccccccaggtgtacacaatcccgccacccaaagaacagatggccaaggacaaggtgtccctgacctgcatgatcaccgatttcttcccagaggatattaccgtggaatggcagtggaacggccagcccgccgagaactacaagaacacccagcctatcatggacaccgacggctcctacttcgtgtactccaagctgaacgtgcagaagtccaactgggaggccggcaacaccttcacctgtagcgtgctgcacgagggcctgcacaatcaccacaccgagaagtccctgtcccactcccctggctag

SEQ ID NO: 41 - DNA, 720 bp, Artificial Sequence
  Nucleotide sequence for VL mAb D
atggagaccgacaccctgctgctctgggtgctgctgctctgggtgcccggctccaccggagacgtggtgatgacccagacacccctgagtctgcccgtgagcttgggcgaccaggccagcatcagctgtaggagctcacagagcctggtgaacagcgacggcaacaccttcctgcagtggctcctgcaaaaacccggccaaagcccgaagctgcttatatacaaggtgagcaataggttcagtggtgtgcccgaccgcttcagcggcagcggtagcggcaccgacttcaccctgaggatcagcagggtggaggccgaggacctgggcgtgtacttctgcagccagagcacccacgtgccctggaccttcggcggaggaaccaaactggaaatcaagcgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 42 - DNA, 1404 bp, Artificial Sequence
  Nucleotide sequence for VH mAb D
atggagaccgacaccctgctgctctgggtgctgctgctctgggtgcccggctccaccggacagatccagctgcaacaaagcggccctgagctggtgaagcccggtgctagcgtgaaggtgagctgtaaggcaagcggctacgccttcacaagttaccagctgtactgggtaaagcaaagccacggcaagagcctggagtggatcggctatatcgacccctacaacggcggcaccggctacaaccagaagttcaagggtaaggccaccttgaccgtggacaagagcagcagcaccgcctacatgcatctgaacagcctgaccagcgaggacagcgccgtgtactactgcgccagccccaggtggcttcccgctggcgactactggggccagggcaccagcgtgactgtgagctctgctaaaacaacagccccatcggtctatccactggcccctgtgtgtggagatacaactggctcctcggtgactctaggatgcctggtcaagggttatttccctgagccagtgaccttgacctggaactctggttccctgtccagtggtgtgcacaccttcccagctgtcctgcagtctgacctctacaccctcagctcaagcgtgactgtaaccagctcgacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtagtcgttgatgtgagcgaggatgacccagatgtccagatcagctggtttgtgaacaacgtggaagtgcacactgctcagacacagacgcatagagaggattacaacagtactctccgggttgtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaggagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggagaagaagaactgggtggagagaaatagctactcctgttcagtggtccacgagggtctgcacaatcaccacacgactaagagcttctcccggactccgggttag